(19) United States
(12) Patent Application Publication
(10) Pub. No.: US 2007/0099931 A1
(43) Pub. Date: May 3, 2007

(54) Pharmaceutical dosage forms and compositions

(75) Inventors: Krishnendu Ghosh, Sparkill, NY (US); Arwinder S. Nagi, Thiells, NY (US); Xiaohong Pan, Highland Mills, NY (US); Melissa Lin, Wanaque, NJ (US); Leonid Linberg, Upper Saddle River, NJ (US); Ping Cai, New City, NY (US); Eric N.C. Browne, Quebec (CA); Michel Bernatchez, Montreal (CA); Mark Lankau, Dollard des Ormeaux (CA); Sangeeta Raje, Audubon, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Wyeth, Madison, NJ (US)

(21) Appl. No.: 11/517,605

(22) Filed: Sep. 6, 2006

Related U.S. Application Data
(63) Continuation-in-part of application No. 11/082,548, filed on Mar. 17, 2005.
(60) Provisional application No. 60/715,417, filed on Sep. 9, 2005.
(60) Provisional application No. 60/554,622, filed on Mar. 19, 2004.

(51) Int. Cl. A61K 31/496

(52) U.S. Cl. 514/253.11

(57) ABSTRACT

The invention relates to, for example, novel formulations and methods for the delivery of 4-cyano-N-{(2R)—2-[4-(2,3-dihydro-benzo[1,4]dioxin-5-yl)-piperazin-1-yl]-propyl}-N-pyridin-2-yl-benzamide, pharmaceutically acceptable salts thereof, structurally related compounds and/or metabolites; as well as to use of these formulations and methods for treating disease.

### CROSS REFERENCE TO RELATED APPLICATIONS

This application claims the benefit of U.S. Application No. 60/715,417, filed Sep. 9, 2005. This application is a continuation-in-part application of U.S. application Ser. No. 11/082,548, filed Mar. 21, 2005, currently pending, which claims the benefit of U.S. Application No. 60/554,622, filed Mar. 19, 2004. The entire disclosures of these applications are incorporated herein by reference.

### FIELD OF THE INVENTION

This invention relates, for example, to novel formulations and methods for the delivery of 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salts thereof, structurally related compounds, and/or metabolites; as well as to use of these formulations and methods for treating disease.

### BACKGROUND OF THE INVENTION

Recent data has suggested that the 5-HT_{1A} receptor is involved in cognitive processing. See, for example, Schechter, L. E., et al., “Lecozotan (SRA-333): A Selective Serotonin 1A Receptor Antagonist That Enhances the Stimulated Release of Glutamate and Acetylcholine in the Hippocampus and Possesses Cognitive-Enhancing Properties,” JPET 314:1274-1289, 2005. The 5-HT_{1A} receptor antagonist, 4-cyano-N-[(2R)-[4-(2,3-dihydrobenzo[1,4]dioxin-5-yl)piperazin-1-yl]propyl]-N-pyridin-2-yl-benzamide hydrochloride (lecozotan) has been characterized in multiple in vitro and in vivo pharmacologic assays as a drug to treat cognitive dysfunction. In vitro binding and intrinsic activity determinations demonstrated that lecozotan is a potent and selective 5-HT_{1A} receptor antagonist. Using in vivo microdialysis, lecozotan (0.3 mg/kg sc) antagonized the decrease in hippocampal extracellular 5-HT induced by a challenge dose (0.3 mg/kg sc) of 8 OH-DPAT and had no effects alone at doses 10-fold higher. Lecozotan significantly potentiated the potassium chloride-stimulated release of glutamate and acetylcholine in the dentate gyrus of the hippocampus. Chronic administration of lecozotan did not induce 5-HT_{1A} receptor tolerance or desensitization in a behavioral model indicative of 5-HT_{1A} receptor function. In drug discrimination studies, lecozotan (0.01-1 mg/kg im) did not substitute for 8-OH-DPAT and produced a dose-related blockade of the 5-HT_{1A} agonist discriminative stimulus cue. In aged rhesus monkeys, lecozotan produced a significant improvement in task performance efficiency at an optimal dose (1 mg/kg po). Learning deficits induced by the glutamatergic antagonist MK-801 (assessed by perceptually complex and visual spatial discrimination) and by specific cholinergic lesions of the hippocampus (assessed by visual spatial discrimination) were reversed by lecozotan (2 mg/kg im) in marmosets. The heterosynaptic nature of the effects of lecozotan imbues this compound with a novel mechanism of action directed at the biochemical pathologies underlying cognitive loss in Alzheimer's disease.

Since 4-cyano-N-{(2R)-2-[4-(2,3-dihydro-benzo[1,4]dioxin-5-yl)-piperazin-1-yl]-propyl}-N-pyridin-2-yl-benzamide (lecozotan) and its pharmaceutically acceptable salts, structurally related compounds, metabolites, and combinations thereof are important in the treatment of Alzheimer's Disease, it is important to provide formulations of these active ingredients that provide optimum bioavailability and efficacy. The invention is directed to these, as well as other, important needs.

### SUMMARY OF THE INVENTION

The present invention provides, inter alia, formulations comprising 4-cyano-N-{(2R)-2-[4-(2,3-dihydro-benzo[1,4]dioxin-5-yl)-piperazin-1-yl]-propyl}-N-pyridin-2-yl-benzamide, also referred to as lecozotan, pharmaceutically acceptable salts thereof, structurally related compounds, metabolites, and combinations thereof.

Compounds provided by the present invention include:

4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof (e.g., 4-{(2R)-2-[4-(2,3-dihydro-benzo[1,4]dioxin-5-yl)-piperazin-1-yl]-propyl}-N-pyridin-2-yl-benzamide hydrochloride salt) and structurally related compounds and metabolites thereof, including, but not limited to:

{(2R)-2-[4-(2,3-dihydro-benzo[1,4]dioxin-5-yl)-piperazin-1-yl]-propyl}-N-pyridin-2-yl-amine or a pharmaceutically acceptable salt thereof; 4-cyano-N-{(2S)-2-[4-(2,3-dihydro-benzo[1,4]dioxin-5-yl)-piperazin-1-yl]-propyl}-N-pyridin-2-yl-benzamide or a pharmaceutically acceptable salt thereof;

4-cyano-N-(2-piperazin-1-yl-propyl)-N-pyridin-2-yl-benzamide or a pharmaceutically acceptable salt thereof; N-(5-chloro-pyridin-2-yl)-4-cyano-N-[2-(4-hydroxy-piperazin-1-yl)-propyl]-benzamide or a pharmaceutically acceptable salt thereof;

N-(5-chloro-pyridin-2-yl)-4-cyano-N-{2-[4-(2,3-dihydro-benzo[1,4]dioxin-5-yl)-piperazin-1-yl]-propyl}-benzamide or a pharmaceutically acceptable salt thereof;

4-cyano-N-{(2R)-2-[4-(8-hydroxy-2,3-dihydro-benzo[1,4]dioxin-5-yl)-piperazin-1-yl]-propyl}-N-pyridin-2-yl-benzamide or a pharmaceutically acceptable salt thereof;

4-cyano-N-{(2R)-2-[4-(3-hydroxy-2,3-dihydro-benzo[1,4]dioxin-5-yl)-piperazin-1-yl]-propyl}-N-pyridin-2-yl-benzamide or a pharmaceutically acceptable salt thereof;

4-cyano-N-{(2R)-2-[4-(2-hydroxy-2,3-dihydro-benzo[1,4]dioxin-5-yl)-piperazin-1-yl]-propyl}-N-pyridin-2-yl-benzamide or a pharmaceutically acceptable salt thereof;

4-cyano-N-(2R-2-piperazin-1-yl-propyl)-N-pyridin-2-yl-benzamide or a pharmaceutically acceptable salt thereof;

4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]-2-methylpropyl}-2,3-dihydro-1,4-benzodioxin-5-yl)piperazin-1-yl]propyl}-N-pyridin-2-ylbenzamide or a pharmaceutically acceptable salt thereof;

4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]butyl}-2,3-dihydro-1,4-benzodioxin-5-yl)piperazin-1-yl]propyl}-N-pyridin-2-ylbenzamide or a pharmaceutically acceptable salt thereof;

4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]hexyl}-2,3-dihydro-1,4-benzodioxin-5-yl)piperazin-1-yl]propyl}-N-pyridin-2-ylbenzamide or a pharmaceutically acceptable salt thereof;

4-cyano-N-{(2R)-2-[4-(8-{[8-(4-{(1S)-2-[(4-cyanobenzoyl)(pyridine-2-yl)amino]-1-methylethyl}piperazin-1-yl)-2,3-dihydro-1,4-benzodioxin-5-yl]methyl}-2,3-dihydro-1,4-benzodioxin-5-yl)piperazin-1-yl]propyl}-N-pyridin-2-ylbenzamide or a pharmaceutically acceptable salt thereof;

4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]ethyl}-2,3-dihydro-1,4-benzodioxin-5-yl)piperazin-1-yl]propyl}-N-pyridin-2-ylbenzamide or a pharmaceutically acceptable salt thereof; and

4-cyano-N-[2(R)—(4-cyano-benzamido)-propyl]-N-pyridin-2-yl-benzamide or a pharmaceutically acceptable salt thereof.

In one embodiment, the compounds are in the form of particles. In one aspect, the particles have a mean diameter of no more than about 20 microns. In another aspect, the particles have a mean diameter of from about 0.75 to about 10 microns. In another aspect, the particles have a mean diameter of from about 2 to about 8 microns.

Compositions of the present invention comprise 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof (e.g., 4-{(2R)-[4-(2,3-dihydro-benzo[1,4]dioxin-5-yl)-piperazin-1-yl]-propyl}-N-pyridin-2-yl-benzamide hydrochloride salt), structurally related compounds or metabolites thereof as described herein. In some embodiments, compositions of the present invention comprise 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof and one or more structurally related compounds and/or metabolites. In some embodiments, 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof and its structurally related compounds and/or metabolites are present in the composition in the form of particles. In one aspect, the particles have a mean diameter of no more than about 20 microns. In another aspect, the particles have a mean diameter of from about 0.75 to about 10 microns. In another aspect, the particles have a mean diameter of from about 2 to about 8 microns. In some embodiments, the structurally related compounds and/or metabolites, when provided in a composition with 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof (e.g., 4-{(2R)-2-[4-(2,3-dihydro-benzo[1,4]dioxin-5-yl)-piperazin-1-yl]-propyl}-N-pyridin-2-yl-benzamide hydrochloride salt) are in an amount of less than about 0.1 weight percent each. In some embodiments, compositions of the present invention further comprise a pharmaceutically acceptable carrier.

In some embodiments, compositions and dosage forms of the present invention comprising 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof are substantially free of one or more dimers of 4-cyano-N-{(2R)-2-[4-(2,3-dihydro-benzo[1,4]dioxin-5-yl)-piperazin-1-yl]-propyl}-N-pyridin-2-yl-benzamide. “Substantially free,” as used in this context, means that the dimers will be present in the compositions in an amount of less than about 0.5% each, by weight, preferably in an amount of less than about 0.3% each, by weight, more preferably in an amount of less than about 0.2% each, by weight, and even more preferably in an amount of less than about 0.1% each, by weight, based on the total weight of the composition, and in the dosage forms in an amount of less than about 0.5% each, by weight, preferably in an amount of less than about 0.3% each, by weight, more preferably in an amount of less than about 0.2% each, by weight, and even more preferably in an amount of less than about 0.1% each, by weight, based on the weight of the active ingredient in the dosage form. Accordingly, the present invention provides formulations comprising 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof that are substantially free of dimers of 4-cyano-N-{(2R)-2-[4-(2,3-dihydro-benzo[1,4]dioxin-5-yl)-piperazin-1-yl]-propyl}-N-pyridin-2-yl-benzamide and/or other structurally related compounds of 4-cyano-N-{(2R)-2-[4-(2,3-dihydro-benzo[1,4]dioxin-5-yl)-piperazin-1-yl]-propyl}-N-pyridin-2-yl-benzamide. Representative dimers of 4-cyano-N-{(2R)-2-[4-(2,3-dihydro-benzo[1,4]dioxin-5-yl)-piperazin-1-yl]-propyl}-N-pyridin-2-yl-benzamide are as shown in formulas 7 and 8.

Dosage forms of the present invention comprise 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof (e.g., 4-{(2R)-2-[4-(2,3-dihydro-benzo[1,4]dioxin-5-yl)-piperazin-1-yl]-propyl}-N-pyridin-2-yl-benzamide hydrochloride salt), structurally related compounds or metabolites as described herein. In some embodiments, dosage forms of the present invention will comprise 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof and one or more structurally related compounds and/or metabolites. In some embodiments, 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof and its structurally related compounds and/or metabolites are present in the dosage form in the form of particles. In one aspect, the particles have a mean diameter of no more than about 20 microns. In another aspect, the particles have a mean diameter of from 0.75 to about 10 microns. In another aspect, the particles have a mean diameter of from about 2 to about 8 microns. In some embodiments, the structurally related compounds and/or metabolites when provided in a dosage form with 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof (e.g., 4-{(2R)-2-[4-(2,3-dihydro-benzo[1,4]dioxin-5-yl)-piperazin-1-yl]-propyl}-N-pyridin-2-yl-benzamide hydrochloride salt) is in an amount of less than about 0.1%, by weight, based on the total weight of the dosage form.

The term active ingredient refers to 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof (e.g., 4-{(2R)-2-[4-(2,3-dihydro-benzo[1,4]dioxin-5-yl)-piperazin-1-yl]-propyl}-N-pyridin-2-yl-benzamide hydrochloride salt), structurally related compounds or metabolites (as shown herein) and their pharmaceutically acceptable salts.

In some embodiments, pharmaceutical compositions and/or dosage forms comprise in addition to the active ingredient (e.g., 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof) at least one rate controlling polymer and at least one organic acid. In some embodiments, the organic acid is citric acid anyhydrate, citric acid monohydrate, ascorbic acid, aspartic acid, glutamic acid, fumaric acid, malic acid or tartaric acid. In some embodiments, the organic acid is citric acid or a polyfunctional organic acid. In some embodiments, the at least one release rate controlling polymer is a methylcellulose. In some embodiments, the polymer is a hydroxypropyl methylcellulose, hydroxypropyl cellulose, hydroxyethyl cellulose or hydroxypropyl methylcellulose phthalate. In some embodiments, the hydroxypropyl methylcellulose is hypromellose 2208 or 2910 (e.g., Methocel™ K4M, Methocel™ K15M, Methocel™ K100M, Methocel™ E10M, Methocel™ E4M, Methocel™ K100LV, Methocel™ E50LV, Methocel™ E5, Methocel™ E6, or Methocel™ E15LV. In some embodiments, the organic acid is citric acid and the rate controlling polymer is hypromellose 2208, (e.g., Methocel™ K4M premium CR and/or Methocel™ K100M Premium CR).

In some embodiments, pharmaceutical compositions and/or dosage forms further comprise at least one filler. In some embodiments, the filler is microcrystalline cellulose, lactose, calcium carbonate, calcium phosphate, maltodextrin, dextrose, fructose, maltose, mannitol, starch, or sucrose. In some embodiments, the microcrystalline cellulose is silicified microcrystalline cellulose and the lactose is lactose monohydrate. In some embodiments, pharmaceutical compositions and/or dosage forms further comprise at least one lubricant. In some embodiments, the lubricant is magnesium stearate, talc, stearic acid, or colloidal silicon dioxide. Accordingly in some embodiments, pharmaceutical compositions and/or dosage forms of the present invention comprise, in addition to the active ingredient or ingredients, at least one rate controlling polymer, at least one organic acid, at least one filler, and at least one lubricant.

In some embodiments, pharmaceutical compositions and/or dosage forms of the present invention comprise about 2 to about 45 or 46 parts of a release rate controlling polymer and about 1 to about 5 parts of an organic acid per part of active ingredient. In some embodiments, the pharmaceutical compositions and/or dosage forms comprise about 0.4 to about 10 mg of active ingredient. In some embodiments, the pharmaceutical compositions and/or dosage forms of the present invention comprise about 50 to about 150 mg of rate controlling polymer(s), about 5 to about 50 mg of organic acid(s), about 85 to about 179 mg of filler(s) and about 1 mg of lubricant. In some embodiments, there is from about 2 to about 50 mg of organic acid(s).

In some embodiments, pharmaceutical compositions and/or dosage forms of the present invention comprise in addition to the active ingredient (e.g., 4-cyano-N-{(2R)-[4-(2,3-dihydro-benzo[1,4]dioxin-5-yl)-piperazin-1-yl]-propyl}-N-pyridin-2-yl-benzamide or a pharmaceutically acceptable salt form thereof), at least one filler and at least one lubricant. In some embodiments, the filler is microcrystalline cellulose, lactose, calcium carbonate, calcium phosphate, maltodextrin, dextrose, fructose, maltose, mannitol, starch, sucrose or a blend thereof. In some embodiments, the filler is microcrystalline cellulose, lactose, or a blend thereof. In some embodiments, pharmaceutical compositions and/or dosage forms further comprise at least one lubricant. In some embodiments, the lubricant is magnesium stearate, talc, stearic acid, or colloidal silicon dioxide. In some embodiments, the lubricant is magnesium stearate.

In some embodiments, pharmaceutical compositions and/or dosage forms comprise about 15 to about 300 parts of filler and about 0.1 to about 3 parts of lubricant per part of active ingredient. In some embodiments, the pharmaceutical compositions and/or dosage forms comprise about 0.1 to about 5 mg of 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt form thereof. In some embodiments, the pharmaceutical compositions and/or dosage forms comprise about 80 to about 150 mg of one or more filler(s) and at least about 0.75 mg of one or more lubricant(s).

In some embodiments, the dosage forms of the present invention are in the form of tablets. In one aspect, the tablets are film coated.

In some embodiments, the compositions or dosage forms of the present invention are in the form of a dry blend.

The present invention provides processes of providing the compositions and dosage forms of the present invention. In some embodiments, the compositions are compressed for a time and under conditions effective to form a tablet thereof. In some embodiments, the tablets are further film coated.

The present invention also provides processes comprising mixing the active ingredient, at least one rate controlling polymer and at least one organic acid thereby forming a blend thereof. In some embodiments, the process further comprises compressing the blend for a time and under conditions effective to form a tablet thereof. In some embodiments, the tablets are further film coated.

The present invention also provides processes comprising mixing the active ingredient, at least one filler and at least lubricant thereby forming a blend thereof. In some embodiments, the process further comprises compressing the blend for a time and under conditions effective to form a tablet thereof. In some embodiments, the tablets are further film coated.

In some embodiments, the dosage forms of the present invention are free of base.

In some embodiments, the present invention provides methods and processes of administering a dosage form, compound or composition of the present invention to a mammal, e.g., to a human. In some embodiments, the dosage forms, compounds, or compositions are orally administered. In one aspect, they are orally administered once every 12 or 24 hours. In another aspect, they are orally administered once every 48 hours. In some particularly preferred embodiments, the dosage forms, compounds, or compositions are administered to treat Alzheimer's Disease.

In certain aspects, the present invention provides methods of administering to a patient an oral dosage form that comprises lecozotan and a pharmaceutically acceptable carrier. In some embodiments, the methods comprise administering to a patient an oral dosage form that comprises lecozotan and a pharmaceutically acceptable carrier and achieves a maximum plasma concentration (Cₘₐₓ) in a patient and a 24 hour plasma concentration (C₂₄) in a patient, wherein a mean ratio of Cₘₐₓ/C₂₄ in a patient population is from about 5:1 to about 1.1:1. In certain embodiments, the mean ratio of Cₘₐₓ/C₂₄ is from about 3:1 to about 1.1:1, from about 2.8:1 to about 1.1:1, or from about 2.3:1 to about 1.1:1. In certain aspects, the Cₘₐₓ and C₂₄ values are measured after administration of a single dose of the oral dosage form to a patient. In some embodiments, the oral dosage form comprises about 5 mg of lecozotan. In certain aspects, the mean tₘₐₓ in the patient population is about 3.5 hours or greater or about 5 hours or greater. In certain aspects, the mean Cₘₐₓ in the patient population is about 100 ng/ml or lower. In some embodiments, administration of these dosage forms minimizes an adverse side effect associated with the administration of lecozotan to a patient. In some embodiments, these methods include a step of identifying a patient at risk of an adverse side effect through administration of lecozotan; and subsequently administering the oral dosage form to the patient. In certain aspects, the adverse side effect is headache, dizziness, paresthesia, abnormal vision, tinnitus, or a combination thereof.

In some embodiments, the methods of administering lecozotan and a pharmaceutically acceptable carrier comprise administering to a patient an oral dosage form that comprises lecozotan and a pharmaceutically acceptable carrier and achieves a maximum plasma concentration (Cₘₐₓ) in a patient and a 24 hour plasma concentration (C₂₄) in a patient, wherein a mean ratio of Cₘₐₓ/C₂₄ in a patient population is from about 2.4:1 to about 1.1:1. In certain embodiments, the mean ratio of Cₘₐₓ/C₂₄ is from about 2.3:1 to about 1.1:1, from about 2:1 to about 1.1:1, from about 1.9:1 to about 1.1:1 or from about 1.5:1 to about 1.1:1. In certain aspects, the Cₘₐₓ and C₂₄ values are measured at steady state in a fasting population. In some embodiments, about 10 mg of lecozotan is provided daily to the patient. In certain aspects, the mean Cₘₐₓ in the patient population is about 350 ng/ml. In some embodiments, administration of these dosage forms minimizes an adverse side effect associated with the administration of lecozotan to a patient. In some embodiments, these methods include a step of identifying a patient at risk of an adverse side effect through administration of lecozotan; and subsequently administering the oral dosage form to the patient. In certain aspects, the adverse side effect is headache, dizziness, paresthesia, abnormal vision, tinnitus, or a combination thereof.

The present invention also provides methods comprising administering to a patient an oral dosage form that comprises lecozotan and a pharmaceutically acceptable carrier and achieves a mean maximum plasma concentration (Cₘₐₓ) in a patient population and a mean 24 hour plasma concentration (C₂₄) in a patient population, wherein a ratio of mean Cₘₐₓ/mean C₂₄ is from about 5:1 to about 0.5:1. In certain aspects the ratio of mean Cₘₐₓ/mean C₂₄ is from about 2.8:1 to about 1.1:1, from about 2:1 to about 1.1:1, or from about 1.5:1 to about 1.1:1. In certain aspects, the Cₘₐₓ and C₂₄ values are measured after administration of a single dose of the oral dosage form to a patient. In other aspects, the Cₘₐₓ and C₂₄ values are measured at steady state in a fasting population. In some embodiments, the oral dosage form comprises about 5 mg of lecozotan. In some embodiments, administration of these dosage forms minimizes an adverse side effect associated with the administration of lecozotan to a patient. In some embodiments, these methods include a step of identifying a patient at risk of an adverse side effect through administration of lecozotan; and subsequently administering the oral dosage form to the patient. In certain aspects, the adverse side effect is headache, dizziness, paresthesia, abnormal vision, tinnitus, or a combination thereof.

The present invention also provides methods comprising administering to a patient an oral dosage form that comprises lecozotan and a pharmaceutically acceptable carrier and achieves a mean maximum plasma concentration (Cₘₐₓ) in a patient population and a mean 12 hour plasma concentration (C₁₂) in a patient population, wherein a ratio of mean Cₘₐₓ/mean C₂₄ is from about 3:1 to about 0.5:1. In certain embodiments, the ratio of mean Cₘₐₓ/mean C₁₂ is from about 2:1 to about 1.1:1 or from about 1.5:1 to about 1.1:1. In some embodiments, administration of these dosage forms minimizes an adverse side effect associated with the administration of lecozotan to a patient. In some embodiments, these methods include a step of identifying a patient at risk of an adverse side effect through administration of lecozotan; and subsequently administering the oral dosage form to the patient. In certain aspects, the adverse side effect is headache, dizziness, paresthesia, abnormal vision, tinnitus, or a combination thereof.

In some embodiments, the present invention provides methods for administering lecozotan to a patient comprising administering to the patient an oral dosage form that comprises lecozotan and a pharmaceutically acceptable carrier, wherein the dosage form exhibits an in vitro dissolution profile when measured in a USP type II dissolution apparatus at 50 rpm, in 900 ml of USP pH 6.8 phosphate buffer at 37° C. and a sinker is used for each dosage form, wherein no more than 40% of lecozotan is released at about 2 hours of measurement in said apparatus; and from about 50% to about 85% of lecozotan is released at about twelve hours of measurement in said apparatus.

In some embodiments, the present invention provides methods for administering lecozotan to a patient comprising administering an oral dosage form that comprises lecozotan and a pharmaceutically acceptable carrier to a patient and (i) achieves a mean Cₘₐₓ in a patient population that is less than the mean Cₘₐₓ obtained from administering an equivalent dose of lecozotan from an immediate release formulation to the patient population; (ii) achieves a mean tₘₐₓ in a patient population that is greater than the tₘₐₓ obtained from administering an equivalent dose of lecozotan from an immediate release formulation to the patient population; and (iii) achieves a curve of concentration of lecozotan over time, the curve having an area under the curve (AUC) in a patient population that is substantially the same as the AUC obtained from administering an equivalent dose of lecozotan from an immediate release dosage form to the patient population. In certain embodiments, the oral dosage form achieves a mean Cₘₐₓ of about 100 ng/ml or less and/or a mean tₘₐₓ of from about 4 hours to about 8 hours and/or a mean AUC that is from about 2000 to about 2500 ng h/mL. In one aspect, these measurements are measured after administration of a single dose of the oral dosage form, for example, a single dose of 5 mg of lecozotan. In certain embodiments, the oral dosage form achieves a mean Cₘₐₓ of less than 400 ng/mL, for example a mean Cₘₐₓ of about 350 ng/mL, and/or a mean tₘₐₓ of from about 4 hours to about 8 hours and/or a mean AUC that is from about 5500 to about 6300 ng h/mL. In certain aspects, these measurements are measured at steady state in a fasting population. In certain aspects, 10 mg of lecozotan is administered daily to the patient. In some embodiments, administration of these dosage forms minimizes an adverse side effect associated with the administration of lecozotan to a patient. In some embodiments, these methods include a step of identifying a patient at risk of an adverse side effect through administration of lecozotan; and subsequently administering the oral dosage form to the patient. In certain aspects, the adverse side effect is headache, dizziness, paresthesia, abnormal vision, tinnitus, or a combination thereof.

### BRIEF DESCRIPTION OF THE DRAWINGS

FIG. 1 presents a graph showing mean lecozotan concentration over time for three sustained release formulations and one immediate release formulation after a single dose of 5 mg of lecozotan.

FIG. 2 presents a graph showing mean lecozotan plasma concentration versus time profiles in Alzheimer's patients receiving multiple oral doses of immediate release (5 mg twice daily) and sustained release (10 mg once daily) formulations.

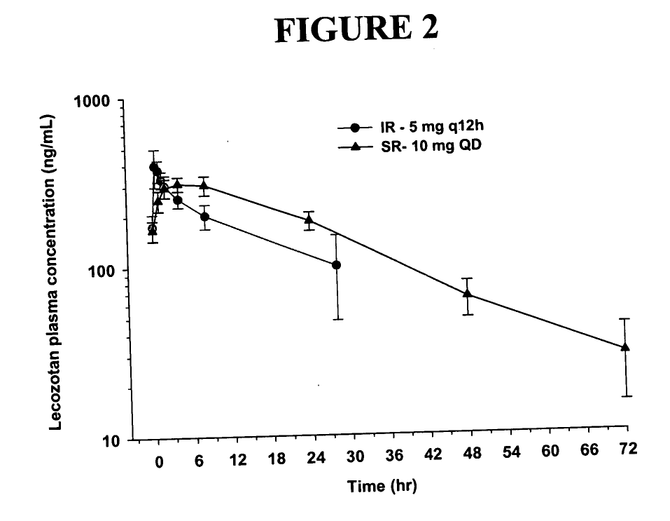

FIG. 3 presents a graph showing lecozotan plasma concentration vs time profile in Alzheimer's patients receiving single and multiple oral doses (QD) doses of 10 mg lecozotan sustained release formulation as described in part 2 of example 8. The data for Day 1 shows from 0 to 24 hours. The data for Day 28 shows 0 to 72 hours.

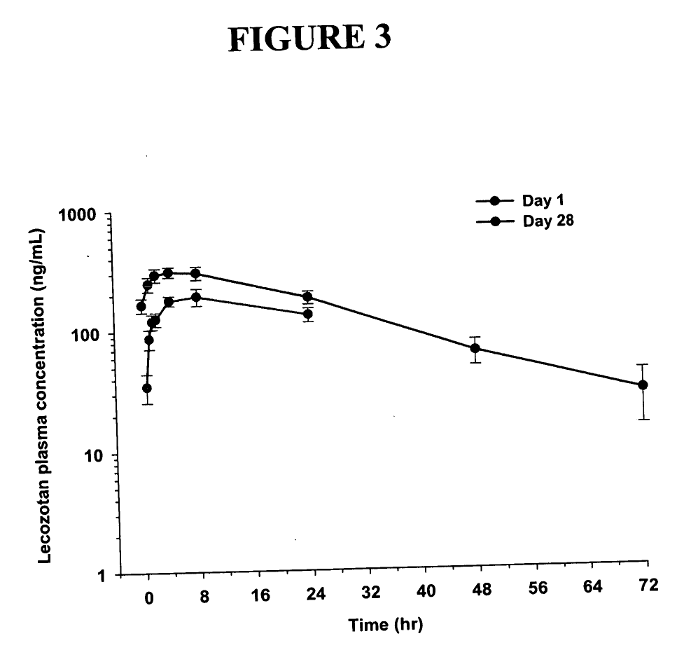

FIG. 4 presents a graph showing a comparison of lecozotan mean plasma concentrations vs. time profiles in Alzheimer's patients receiving multiple oral doses of IR (5 mg) and SR (10 mg QD) formulations.

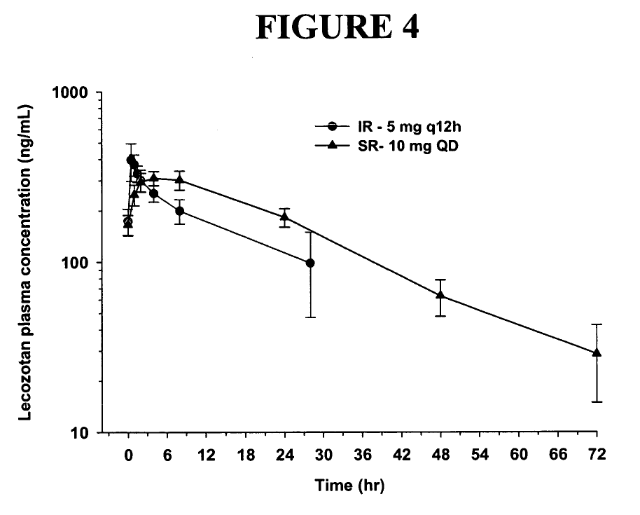

### DETAILED DESCRIPTION OF THE INVENTION

The present invention provides, inter alia, formulations comprising 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salts thereof, structurally related compounds, and/or metabolites. As used herein, the term “formulations” refers to compounds, compositions, and dosage forms, such as, for example, immediate release and sustained release dosage forms.

The present invention also provides processes for making the formulations and methods of administering them to a mammal.

Preferred formulations for use in the present invention are those that act as serotonergic agents and have 5-HT_{1A} binding activity. In particular, preferred compounds act as 5-HT_{1A} antagonists. See, for example, U.S. Pat. No. 6,784,294, U.S. Pat. No. 6,713,626, U.S. Pat. No. 6,469,007, U.S. Pat. No. 6,586,436, U.S. Pat. No. 5,710,149, and U.S. Pat. No. 6,127,357, and WO 97/03982, the disclosures of which are incorporated herein by reference in their entirety for all purposes. Compounds of the present invention, as well as compositions comprising more than one compound of the present invention, can be prepared by those skilled in the art of organic synthesis employing known methods that utilize readily available reagents and starting materials, see, for example, EP-B-0512755, WO 97/03982, U.S. Pat. No. 6,127,357, U.S. Pat. No. 6,469,007, U.S. Pat. No. 6,713,626, and U.S. Pat. No. 6,784,294, and U.S. Pat. No. 2,003,0208075A1, the disclosures of which are incorporated herein by reference in their entirety for all purposes.

Such methods include alkylating 1-(2,3-dihydro-1,4-benzodioxin-5-yl)piperazine hydrochloride with sulfamate 4,5-dihydro-5S-methyl-3-(2-pyridinyl)-3H[1.2.3]oxathiazole-2,2-dioxide to give a sulfamic acid intermediate which is hydrolyzed to {(2R)-2-[4-(2,3-dihydro-benzo[1,4]dioxin-5-yl)-piperazin-1-yl]-propyl}pyridin-2-yl-amine and then treating {(2R)-2-[4-(2,3-dihydro-benzo[1,4]dioxin-5-yl)-piperazin-1-yl]-propyl}pyridin-2-yl-amine with 4-cyanobenzoyl chloride to give 4-cyano-N-{(2R)-2-[4-(2,3-dihydro-benzo[1,4]dioxin-5-yl)-piperazin-1-yl]-propyl}-N-pyridin-2-yl-benzamide base. Treatment of 4-cyano-N-{(2R)-2-[4-(2,3-dihydro-benzo[1,4]dioxin-5-yl)-piperazin-1-yl]-propyl}-N-pyridin-2-yl-benzamide base with hydrochloric acid gives its hydrochloride salt.

In some embodiments of the present invention, preparations comprising 4-cyano-N-{(2R)-2-[4-(2,3-dihydro-benzo[1,4]dioxin-5-yl)-piperazin-1-yl]-propyl}-N-pyridin-2-yl-benzamide and its pharmaceutically acceptable salts are further processed and purified. For example, in one embodiment, a preparation comprising p4-cyano-N-{(2R)-2-[4-(2,3-dihydro-benzo[1,4]dioxin-5-yl)-piperazin-1-yl]-propyl}-N-pyridin-2-yl-benzamide prepared by methods disclosed herein is dissolved in organic solvent, treated with silica gel, and filtered in order to remove structurally related compounds, e.g., dimers represented by Formulas 7 and 8. The remaining product can then be concentrated and re-crystallized in order to provide, e.g., 4-cyano-N-{(2R)-2-[4-(2,3-dihydro-benzo[1,4]dioxin-5-yl)-piperazin-1-yl]-propyl}-N-pyridin-2-yl-benzamide hydrochloride salt.

Preferred formulations of the present invention can be used to modulate, e.g., antagonize or agonize, 5-HT_{1A} receptor activity and are useful in the treatment of diseases such as CNS disorders, including, but not limited to, schizophrenia, (and other psychotic disorders such as paranoia and mano-depressive illness), Parkinson's disease and other motor disorders, anxiety (e.g., generalized anxiety disorders, panic attacks, and obsessive compulsive disorders), depression (such as by the potentiation of serotonin reuptake inhibitors and serotonin norepinephrine reuptake inhibitors), Alzheimer's disease, Tourette's syndrome, migraine, autism, attention deficit disorders and hyperactivity disorders. Preferred formulations are useful for the treatment of sleep disorders, social phobias, pain, thermoregulatory disorders, endocrine disorders, urinary incontinence, vasospasm, stroke, eating disorders such as for example obesity, anorexia and bulimia, sexual dysfunction, and the treatment of alcohol, drug and nicotine withdrawal.

Preferred formulations of the present invention are also useful for the treatment of cognitive dysfunction including but not limited to cognitive dysfunction associated with mild cognitive impairment (MCI), Alzheimer's disease and other dementias including Lewy Body, vascular, and post stroke dementias. Cognitive dysfunction associated with surgical procedures, traumatic brain injury or stroke can also be treated in accordance with the present invention. Further, preferred formulations are useful for the treatment of diseases in which cognitive dysfunction is a co-morbidity such as, for example, Parkinson's disease, autism and attention deficit disorders.

Despite its high solubility in water (about 51 mg/ml at 25° C.), 4-cyano-N-{(2R)-2-[4-(2,3-dihydro-benzo[1,4]dioxin-5-yl)-piperazin-1-yl]-propyl}-N-pyridin-2-yl-benzamide and its salts are preferably provided in micronized form. As such, the present invention provides formulations comprising 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salts thereof, structurally related compounds, or metabolites in micronized and in non-micronized form. For purposes of the present invention, a compound in micronized form is in the form of particles having a mean diameter of no more than about 20 microns. It is understood that compounds of the present invention can be in the form of particles having a mean diameter of greater than about 20 microns, for example in the form of particles having a mean diameter from about 20 microns to about 300 or about 500 microns. Preferably, the particles have a mean diameter of about 10 microns, more preferably a mean diameter from about 0.75 to about 10 microns, even more preferably from about 2 to about 8 microns. Methods of micronization or particle size reduction are known and are thus not described herein in detail.

As will be recognized, 4-cyano-N-{(2R)-2-[4-(2,3-dihydro-benzo[1,4]dioxin-5-yl)-piperazin-1-yl]-propyl}-N-pyridin-2-yl-benzamide is represented by the following formula:

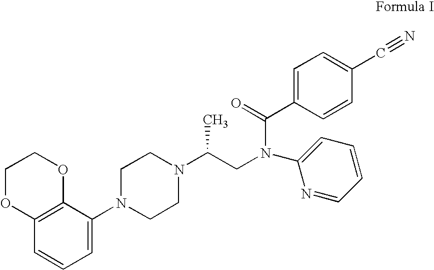

Within the present invention, the compounds of formula 1 can be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include, for example, inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

In certain embodiments, formulations comprising 4-cyano-N-{(2R)-2-[4-(2,3-dihydro-benzo[1,4]dioxin-5-yl)-piperazin-1-yl]-propyl}-N-pyridin-2-yl-benzamide or pharmaceutically acceptable salts thereof will also comprise one or more structurally related compounds that can be detected and quantified using known methods. Examples of such structurally related compounds include, but are not limited to, those compounds represented by Formulas 2-9 and pharmaceutically acceptable salts thereof, including, for example:

{(2R)-2-[4-(2,3-dihydro-benzo[1,4]dioxin-5-yl)-piperazin-1-yl]-propyl}-N-pyridin-2-yl-amine or a pharmaceutically acceptable salt thereof;

4-cyano-N-{(2S)-2-[4-(2,3-dihydro-benzo[1,4]dioxin-5-yl)-piperazin-1-yl]-propyl}-N-pyridin-2-yl-benzamide or a pharmaceutically acceptable salt thereof;

4-cyano-N-(2-piperazin-1-yl-propyl)-N-pyridin-2-yl-benzamide or a pharmaceutically acceptable salt thereof;

4-cyano-N-[(2R)-2-piperazin-1-yl-propyl]-N-pyridin-2-yl-benzamide or a pharmaceutically acceptable salt thereof;

N-(5-chloro-pyridin-2-yl)-4-cyano-N-[2-(4-hydroxy-piperazin-1-yl)-propyl]-benzamide or a pharmaceutically acceptable salt thereof;

N-(5-chloro-pyridin-2-yl)-4-cyano-N-[(2R)-2-(4-hydroxy-piperazin-1-yl)-propyl]-benzamide or a pharmaceutically acceptable salt thereof;

N-(5-chloro-pyridin-2-yl)-4-cyano-N-{2-[4-(2,3-dihydro-benzo[1,4]dioxin-5-yl)-piperazin-1-yl]-propyl}-benzamide or a pharmaceutically acceptable salt thereof;

N-(5-chloro-pyridin-2-yl)-4-cyano-N-{(2R)-2-[4-(2,3-dihydro-1,4-benzodioxin-5-yl)-piperazin-1-yl]-propyl}benzamide or a pharmaceutically acceptable salt thereof;

4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]-2-methylpropyl}-2,3-dihydro-1,4-benzodioxin-5-yl)piperazin-1-yl]propyl}-N-pyridin-2-ylbenzamide or a pharmaceutically acceptable salt thereof;

4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]butyl}-2,3-dihydro-1,4-benzodioxin-5-yl)piperazin-1-yl]propyl}-N-pyridin-2-ylbenzamide or a pharmaceutically acceptable salt thereof;

4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]hexyl}-2,3-dihydro-1,4-benzodioxin-5-yl)piperazin-1-yl]propyl}-N-pyridin-2-ylbenzamide or a pharmaceutically acceptable salt thereof;

4-cyano-N-{(2R)-2-[4-(8-{[8-(4-{(1S)-2-[(4-cyanobenzoyl)(pyridine-2-yl)amino]-1-methylethyl}piperazin-1-yl)-2,3-dihydro-1,4-benzodioxin-5-yl]methyl}-2,3-dihydro-1,4-benzodioxin-5-yl)piperazin-1-yl]propyl}-N-pyridin-2-ylbenzamide or a pharmaceutically acceptable salt thereof;

4-cyano-N-{(2R)-2-[4-(8-{1-[8-(4-{(1S)-2-[(4-cyanobenzoyl)(pyridine-2-yl)amino]-1-methylethyl}piperazin-1-yl)-2,3-dihydro-1,4-benzodioxin-5-yl]ethyl}-2,3-dihydro-1,4-benzodioxin-5-yl)piperazin-1-yl]propyl}-N-pyridin-2-ylbenzamide or a pharmaceutically acceptable salt thereof; and

4-cyano-N-[2(R)—(4-cyano-benzamido)-propyl]-N-pyridin-2-yl-benzamide or a pharmaceutically acceptable salt thereof. 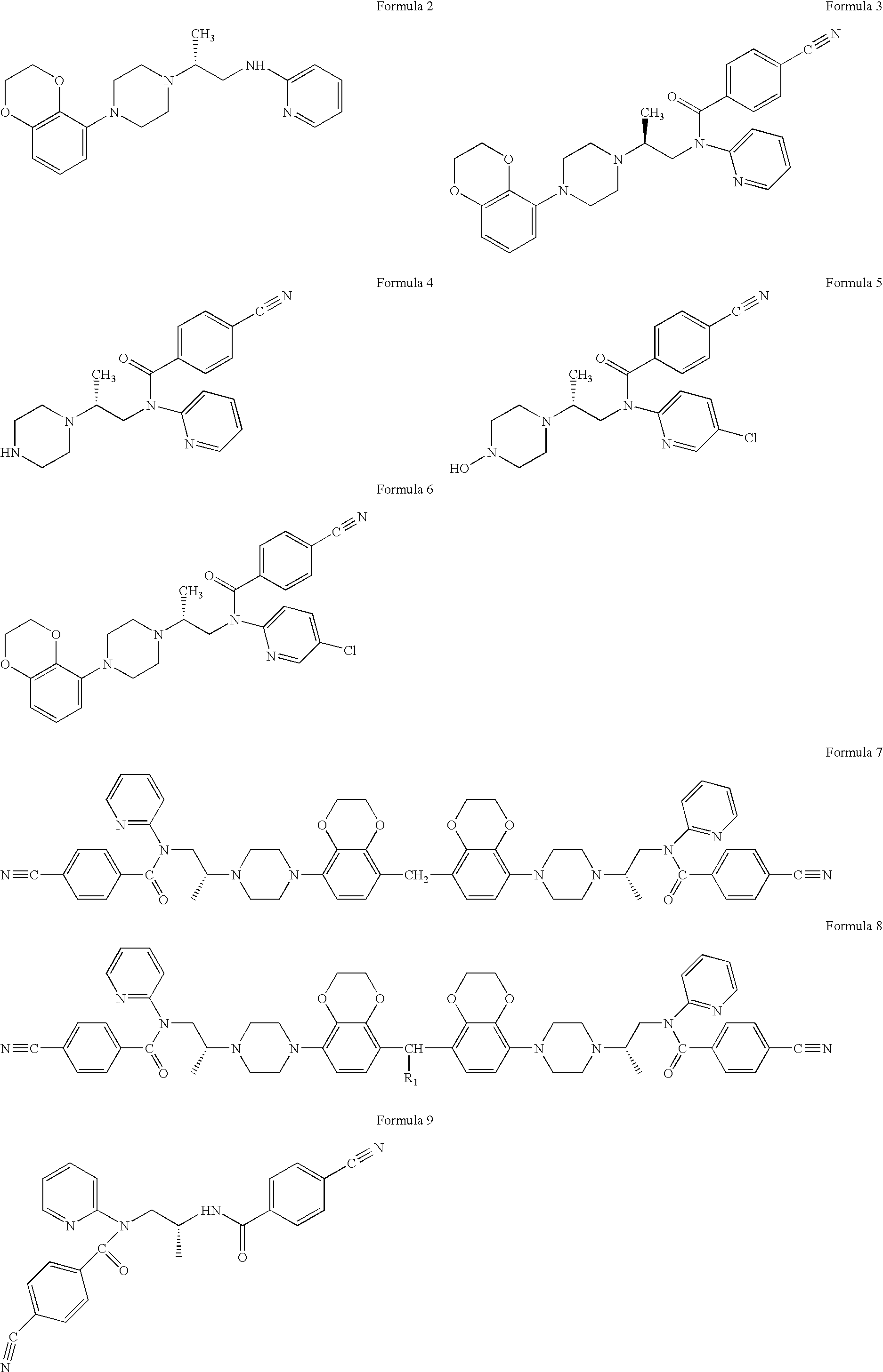
wherein R₁ is —CH₃, —CH(CH₃)₂, —CH₂CH₂CH₃, CH₂CH₂CH₂CH₃ or —CH₂CH₂CH₂CH₂CH₃.

In some embodiments, the present invention provides formulations comprising one or more compounds represented by Formulas 2, 3, 4, 5, 6, 7, 8 or 9 or a pharmaceutically acceptable salt thereof. In some aspects of the present invention, the formulations will comprise 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof and one or more compounds of Formula 2, 3, 4, 5, 6, 7, 8 or 9 or a pharmaceutically acceptable salt thereof. In some embodiments, for example, formulations of the present invention can comprise 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof, {(2R)-2-[4-(2,3-dihydro-benzo[1,4]dioxin-5-yl)-piperazin-1-yl]-propyl}-N-pyridin-2-yl-amine or a pharmaceutically acceptable salt thereof, and 4-cyano-N-{(2S)-2-[4-(2,3-dihydro-benzo[1,4]dioxin-5-yl)-piperazin-1-yl]-propyl}-N-pyridin-2-yl-benzamide or a pharmaceutically acceptable salt thereof. Accordingly, the present invention provides formulations comprising 4-cyano-N-{(2R)-2-[4-(2,3-dihydro-benzo[1,4]dioxin-5-yl)-piperazin-1-yl]-propyl}-N-pyridin-2-yl-benzamide hydrochloride salt, {(2R)-2-[4-(2,3-dihydro-benzo[1,4]dioxin-5-yl)-piperazin-1-yl]-propyl}-N-pyridin-2-yl-amine, and 4-cyano-N-{(2S)-2-[4-(2,3-dihydro-benzo[1,4]dioxin-5-yl)-piperazin-1-yl]-propyl}-N-pyridin-2-yl-benzamide hydrochloride salt. When the structurally related compounds described above are present in combination with 4-cyano-N-{(2R)-2-[4-(2,3-dihydro-benzo[1,4]dioxin-5-yl)-piperazin-1-yl]-propyl}-N-pyridin-2-yl-benzamide or a pharmaceutically acceptable salt thereof, the former preferably predominates and the latter preferably are present in the composition in amount of less than about 10%, more preferably present in amount of less than about 5% and even more preferably in amounts of less than about 1% or 0.1%, for example, in amounts from between about 0.08% and about 0.27%.

4-Cyano-N-{2-[4-(2,3-dihydro-benzo[1,4]dioxin-5-yl)-piperazin-1-yl]-propyl}-N-pyridin-2-yl-benzamide contains one chiral center and is used predominately as the R-isomer. The formulations, e.g., compounds, compositions, or dosage forms, of the present invention can include both R and S isomers, and are not limited to a single enantiomer or particular enantiomeric mixture.

The present invention also provides formulations comprising metabolites of 4-cyano-N-{(2R)-2-[4-(2,3-dihydro-benzo[1,4]dioxin-5-yl)-piperazin-1-yl]-propyl}-N-pyridin-2-yl-benzamide. Metabolites include, but are not limited to, 4-cyano-N-{(2R)-2-[4-(8-hydroxy-2,3-dihydro-benzo[1,4]dioxin-5-yl)-piperazin-1-yl]-propyl}-N-pyridin-2-yl-benzamide or a pharmaceutically acceptable salt thereof, 4-cyano-N-{(2R)-2-[4-(3-hydroxy-2,3-dihydro-benzo[1,4]dioxin-5-yl)-piperazin-1-yl]-propyl}-N-pyridin-2-yl-benzamide or a pharmaceutically acceptable salt thereof, 4-Cyano-N-{(2R)-2-[4-(2-hydroxy-2,3-dihydro-benzo[1,4]dioxin-5-yl)-piperazin-1-yl]-propyl}-N-pyridin-2-yl-benzamide or a pharmaceutically acceptable salt thereof, and 4-cyano-N-(2R-2-piperazin-1-yl-propyl)-N-pyridin-2-yl-benzamide or a pharmaceutically acceptable salt thereof. As is recognized, these metabolites are represented by Formulas 10-13. It will be recognized that these metabolites can be employed as pharmaceutically active compounds and in pharmaceutical dosage forms in their own right, alone or in combination with other pharmaceutically active compounds.

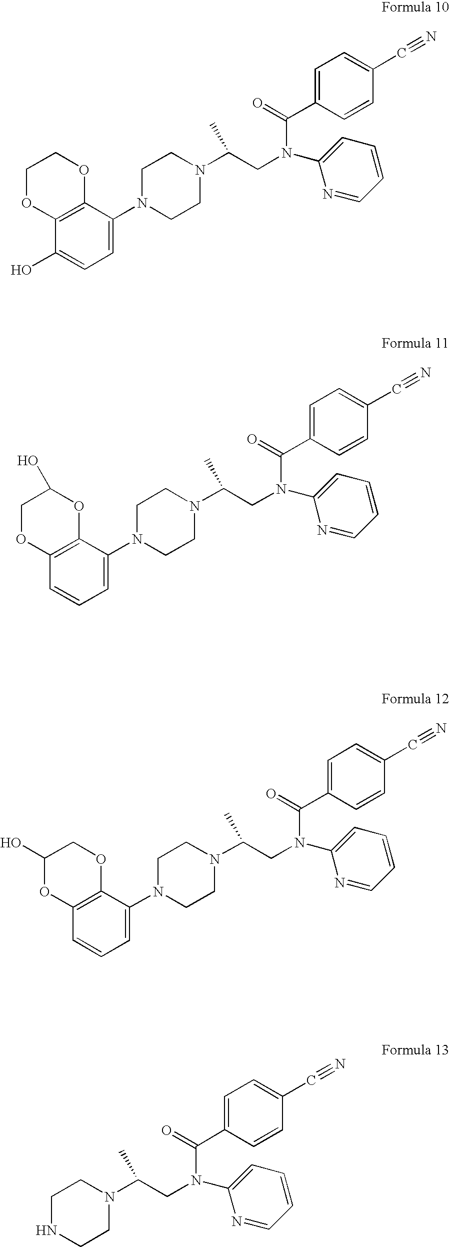

The present invention provides immediate release and sustained release dosage forms comprising one or more active ingredients, e.g., 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salts thereof, structurally related compounds and metabolites thereof that have 5-HT_{1A} binding activity.

A drug “release rate” refers to the quantity of drug released from a dosage form per unit time, e.g., milligrams of drug released per hour (mg/hr). Drug release rates can be calculated, for example, under in vitro dosage form dissolution testing conditions known in the art. As used herein, a drug release rate obtained at a specified time “following administration” refers to the in vitro drug release rate obtained at the specified time following implementation of an appropriate dissolution test. Methods of performing dissolution tests or release rate assays are known in the art. The time at which a specified percentage of the drug within a dosage form has been released can be referenced as the “Tₓ” value, where “x” is the percent of drug that has been released. A commonly used reference measurement for evaluating drug release from oral dosage forms is the time at which 70% or 90% of drug within a dosage form has been released. This measurement is referred to as “T₇₀” or “T₉₀” for the dosage form.

For purposes of this invention, the terms “immediate release formulation” refer to formulations that provide a relatively rapid and non-gradual release of active compound from the formulation; e.g., formulations that contain active compound and a rapidly dissolving carrier that does not retard the release of the active compound from the formulation. Such immediate release formulation are either devoid of release rate controlling polymers or other species that retard the release of the active compound from the formulation, or contain such polymers or species in amounts that are sufficiently small such that the release of the active compound from the formulation is not retarded relative to an otherwise identical formulation lacking such polymers or species. One example of such an immediate release formulation is the active ingredient, e.g., 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salt thereof, structurally related compounds, or metabolites, blended in microcrystalline cellulose, such as Avicel® brand from FMC corporation, which results in greater than 75% dissolution of the active ingredient in less than 0.25 hours in a 0.1 N HCl solution.

As used herein, the terms “sustained release”, “sustained release formulation,” “sustained release dosage formulation” and the like refer to formulations that contain materials that retard the release of active compound from the formulation relative to an “immediate release” formulation as described above, e.g., relative to an otherwise identical formulation lacking the release rate controlling polymer or other release-retarding materials. Thus, the term “sustained release” can apply to any number of extended release forms and is considered substantially synonymous with delayed release, time release, prolonged release, time programmed release, time released, time coated release, sustained release, slow acting, long acting, delayed acting, spaced release, time spaced release, extended acting, extended action, and the like.

The terms “slow release,” “medium release,” and “fast release” are intended to refer to sustained release formulations as described herein that release active compound at a rate that is slow, medium or fast rate relative to each other.

It is appreciated that sustained release formulations can result in a release of active compound from the dosage form at a rate effective to increase the time it takes to reach maximum therapeutic concentration as compared to an immediate release formulation, for example and not limitation, by a period of 50% or more, 100% or more, 150% or more, or 200% or more as compared to an immediate release formulation; e.g., as compared to an otherwise identical formulation lacking the release rate controlling polymer or other release-retarding materials. Sustained release formulations can also result in release of active compound from the dosage form at a rate effective to decrease the maximal therapeutic concentration of said compound compared to an immediate release formulation, for example and not limitation, by at least 10%, at least 20%, at least 25%, at least 30%, at least 40%, or at least 50% compared to an immediate release formulation. Sustained release formulations can also result in release of active compound from the dosage form at a rate effective to increase the amount of time a pharmaceutically effective concentration of the active compound is maintained relative to an immediate release formulation, for example and not limitation, by at least 25%, at least 50%, at least 75%, at least 100%, or at least 125% the amount of time a pharmaceutically effective concentration of active compound is maintained relative to an immediate release formulation. Satisfaction of any of the preceding criteria is sufficient to make a formulation a “sustained release” formulation.

The present invention provides methods for sustained release of the active ingredient comprising administering to a subject the disclosed dosage forms. In one aspect, the release rate of the active compound from the dosage forms is zero order. In another aspect, the release rate of the active ingredient from the dosage forms is ascending.

As used herein, the term “release rate controlling polymer” is intended to denote any polymer material suitable for pharmaceutical dosage forms that retards the release of drug substances from such dosage forms. The release rate-controlling polymer will preferably inhibit the release of the drug in the stomach. Preferably, the release rate-controlling polymer is a hydrogel that imbibes and/or absorbs fluid thereby preventing the release of the drug in the stomach. Examples of suitable release rate controlling polymers can be found in Remington's Pharmaceutical Sciences, 18th Ed., Gennaro, ed., Mack Publishing Co., Easton, Pa., 1990.

Some preferred release rate-controlling polymers suitable for use in the present invention include, without limitation, hydroxypropyl celluloses, methylcelluloses, polymethacrylates, methacrylic acid-methacrylic acid ester copolymers, cellulose acetate phthalate, ethyl celluloses, hydroxyethyl celluloses, hydroxymethyl celluloses, hydroxypropylethyl celluloses, polyvinyl acetate-phthalate, hydroxypropylmethylcellulose phthalate, poly(ethylene) oxides, hydroxypropyl methyl celluloses such as, for example, hypromellose 2208 and 2910 and combinations of two or more thereof. Suitable release rate controlling polymers are available from commercial sources, such as Methocel™ K4M, Methocel™ K15M, Methocel™ K100M, Methocel™ E4M, Methocel™ K100LV, Methocel™ E50LV, Methocel™ E5, Methocel™ E6, Methocel™ E15LV, and Surelease™ available from Colorcon and Eudragit™, RS Eudragit™ RL available from Röhm GmbH & Co. In some embodiments, the formulations of the present invention will comprise high-density matrix-forming hydroxypropyl methylcellulose, low-density matrix-forming hydroxypropyl methylcellulose, or combinations thereof.

The sustained release formulations of the present invention comprise at least one release rate controlling polymer. The range of release rate controlling polymer in the formulation is preferably from about 10% to about 75% by weight, more preferably from about 20% to about 60% by weight. In one embodiment of the present invention, the amount of release rate controlling polymer in a 250 mg dosage form is from about 50 to about 150 mg. In some embodiments, the release rate controlling polymer is a cellulose ether, such as, for example, matrix-forming hydroxypropyl methylcellulose, hydroxypropyl cellulose, or hydroxyethyl cellulose, e.g., Methocel™ K4M Premium CR or Methocel™ K100M Premium CR.

In addition to comprising at least one release rate controlling polymer, sustained release dosage forms of the present invention generally comprise at least one agent to improve the release rate in the intestine, e.g., an organic acid. For uses herein, the term “organic acid” encompasses any acid that can be safely ingested by a mammal. While not wishing to be bound by any particular theory, the acid is believed to improve the release of the drug product in the intestine. Examples of organic acids suitable for use in the present invention include, but are not limited to, tartaric acid, malic acid, fumaric acid, aspartic acid, glutamic acid, glycine hydrochloride, adipic acid, succinic acid, ascorbic acid, oleic acid or citric acid. Preferred organic acids are citric acid or polyfunctional organic acid. The range of organic acid in the formulation is preferably from about 1% to about 30%, more preferably from about 2% to about 10% by weight. In one embodiment of the present invention, the amount of organic acid in a 250 mg dosage form is from about 5 to about 50 mg, preferably from about 5 to about 25 mg. In some embodiments, the amount of organic acid is from about 2 to about 50 mg.

Preferably, the sustained release formulations is substantially free of base. For use herein, a formulation, dosage form, or composition that is substantially of base refers to a formulation, dosage form, or composition that has less than about 10% base, preferably less than about 5% base, and more preferably less than about 1% or 0.1% base. As used herein, the term “base” refers to a chemical compound that functions as a proton acceptor.

In addition to the active compound and release rate controlling polymer, the formulations of the invention can comprise any of a variety of additional materials that confer beneficial properties to the formulation. Such materials include, for example, solubility modifiers such as surfactants such as, for example, sodium lauryl sulfate, acidic compounds, antioxidants, pH modifiers, chelating agents, fillers, disintegrants, binders, lubricants, stabilizers, excipients including water soluble excipients such as sugars and water dispersing excipients such as, for example, microcrystalline cellulose, colloidal silicon dioxide, silicified microcrystalline cellulose and starch. In some embodiments, the formulation is provided at a pH of about 6 or lower, for example at a pH of from about 1 to about 6.

Nonlimiting examples of water-soluble excipients or water dispersing excipients include lactose, mannitol, sucrose, and the like. The water-soluble excipients can be present in a range on weight percentages depending upon the particular therapeutic objective required. For use in the present invention, percentages and parts are expressed as part by weight or percentage by weight, unless otherwise noted. In general, the range of water soluble excipients can be, for example, from about 0% to about 50% or to about 99%, or from about 2% to about 25%. Examples of water dispersible excipients include microcrystalline cellulose, colloidal silicone dioxide, silicified microcrystalline cellulose (Prosolv™), starches, croscarmelose sodium and the like.

Nonlimiting examples of stabilizers include antioxidants such as BHA, BHT, ascorbic acids, tocopherols, and the like. Nonlimiting examples of suitable metal chelators include EDTA, citric acid and the like. Nonlimiting examples of pH modifiers include citric acid, fumaric acid, and the like. Nonlimiting examples of binders include starches, PVP (polyvinylpyrrolidone), HPMC (hydroxypropyl methyl celluloses), HPC (hydroxypropyl cellulose) and the like. Nonlimiting examples of flow aids include magnesium stearate and the like. Nonlimiting examples of solubility modifiers include surfactants like sodium lauryl sulfate or polysorbate (e.g., Tween™ 80), and the like.

In a preferred embodiment, the sustained release formulations of the present invention comprise the active ingredient, at least one release rate controlling polymer, an organic acid, at least one filler and at least one lubricant.

Examples of lubricants include, but are not limited to, stearic acid, magnesium stearate, glyceryl behenate, talc, mineral oil (in PEG), colloidal silicon dioxide and the like. It is appreciated however that any lubricant known in the art can be used in the formulations described herein. The range of lubricant can be, for example, from about 0.2% to about 5%, by weight. In one embodiment of the present invention, the amount of lubricant in a 250 mg dosage form is about 1 mg.

Examples of fillers include, but are not limited to, silicified microcrystalline cellulose, microcrystalline cellulose, cellulose acetate, cellulose diacetate, cellulose triacetate, lactose monohydrate, lactose anhydrous, calcium carbonate, calcium phosphate (e.g., dibasic anhydrous), maltodextrin, dextrose, fructose, maltose, mannitol, starch, starch (e.g., pregelatinized), sucrose, and lactose. It is appreciated, however, that any filler known in the art can be used in the formulations described herein. The range of filler can be, for example, from about 25% to about 75%, or to about 99% by weight. In one embodiment of the present invention (e.g., for exemplary sustained release formulations), the amount of filler present in a 250 mg dosage form is from about 85 to about 179 mg.

The sustained release dosage forms of the present invention can comprise the active compound in any convenient percentage and part in relation to the other ingredients. Typically, the formulation comprises active ingredient in percentage of from about 0.3% to about 25%, preferably from about 0.3% to about 15%. In some embodiments, the formulation will comprise active ingredient in percentage of from about 1% to about 25%, preferably from about 2% to about 15%.

In a preferred embodiment, sustained release formulations will comprise from about 2 to about 46 parts of release relate controlling polymer and about 0.4 to about 10 parts of an agent to improve release rate in the intestine per part active ingredient. More preferably from about 10 to about 46 parts release rate controlling polymer and about 1 to about 5 parts of an agent to improve release rate in the intestine per part active ingredient.

For example, in one embodiment, fast sustained release formulations comprise about 10 parts of release rate controlling polymer, and about 5 parts of organic acid per part of active ingredient, e.g., 4-cyano-N-{(2R)-2-[4-(2,3-dihydro-benzo[1,4]dioxin-5-yl)-piperazin-1-yl]-propyl}-N-pyridin-2-yl-benzamide or pharmaceutically acceptable salt thereof.

In another embodiment, medium sustained release formulations comprise about 25 parts of release rate controlling polymer, and about 5 parts of organic acid per part of active ingredient, e.g., 4-cyano-N-{(2R)-2-[4-(2,3-dihydro-benzo[1,4]dioxin-5-yl)-piperazin-1-yl]-propyl}-N-pyridin-2-yl-benzamide or pharmaceutically acceptable salt thereof.

In another embodiment, slow sustained release formulations comprise about 30 parts of release rate controlling polymer, and about 1 part of organic acid per part of active ingredient, e.g., 4-cyano-N-{(2R)-2-[4-(2,3-dihydro-benzo[1,4]dioxin-5-yl)-piperazin-1-yl]-propyl}-N-pyridin-2-yl-benzamide or pharmaceutically acceptable salt thereof.

In another embodiment, sustained release formulations comprise about 18 parts of release rate controlling polymer, and about 1 part of organic acid per part of active ingredient, e.g., 4-cyano-N-{(2R)-2-[4-(2,3-dihydro-benzo[1,4]dioxin-5-yl)-piperazin-1-yl]-propyl}-N-pyridin-2-yl-benzamide or pharmaceutically acceptable salt thereof.

In another embodiment, sustained release formulations comprise about 46 parts of release rate controlling polymer, and about 1 part of organic acid per part of active ingredient, e.g., 4-cyano-N-{(2R)-2-[4-(2,3-dihydro-benzo[1,4]dioxin-5-yl)-piperazin-1-yl]-propyl}-N-pyridin-2-yl-benzamide or pharmaceutically acceptable salt thereof.

In some embodiments, sustained release formulations comprise about 5 mg of active ingredient, from about 50 to 150 mg of release rate controlling polymer, from about 5 to about 50 mg of organic acid, from about 85 to about 179 mg of filler and about 1 mg of lubricant.

In some embodiments, sustained release formulations comprise about 2 mg of active ingredient, from about 50 to 150 mg of release rate controlling polymer, from about 2 to about 50 mg of organic acid, from about 85 to about 179 mg of filler and about 1 mg of lubricant.

In some embodiments, exemplary sustained release formulations comprise, in a 250 mg tablet, about 5 mg of active ingredient and about 50 mg of release rate controlling polymer. Such an exemplary formulation can further comprise, for example, about 169 mg of filler, about 25 mg of organic acid (or other agent to improve release rate in the intestine) and about 1 mg of lubricant.

In some embodiments, exemplary sustained release formulations comprise, in a 250 mg tablet, about 5 mg of active ingredient and about 125 mg of release rate controlling polymer. Such an exemplary formulation can further comprise, for example, about 94 mg of filler, about 25 mg of organic acid (or other agent to improve release rate in the intestine) and about 1 mg of lubricant.

In some embodiments, exemplary sustained release formulations comprise, in a 250 mg tablet, about 5 mg of active ingredient and about 150 mg of release rate controlling polymer. Such an exemplary formulation can further comprise, for example, about 89 mg of filler, about 5 mg of organic acid (or other agent to improve release rate in the intestine) and about 1 mg of lubricant.

In some embodiments, exemplary sustained release formulations comprise, in a 250 mg tablet, about 5 mg of active ingredient and about 92 mg of release rate controlling polymer. Such an exemplary formulation can further comprise, for example, about 150 mg of filler, about 5 mg of organic acid (or other agent to improve release rate in the intestine) and about 1 mg of lubricant.

In some embodiments, exemplary sustained release formulations comprise, in a 250 mg tablet, about 2 mg of active ingredient and about 92 mg of release rate controlling polymer. Such an exemplary formulation can further comprise, for example, about 150 mg of filler, about 2 mg of organic acid (or other agent to improve release rate in the intestine) and about 1 mg of lubricant.

The sustained release formulations contemplated by the present invention can be in any form suitable for administration to a mammal and are not limited to the examples presented herein.

In some embodiments, the formulations of the invention are in the form of coated pellets or spheres. One nonlimiting example of such a formulation is a sphere containing a core of active compound in an inert matrix, coated with a release rate-controlling polymer as disclosed herein. Nonlimiting examples of suitable release rate controlling polymers are pH dependent or independent polymers described herein, such as polymethacrylates, Eudragit™ IVS, Eudragit™ RS/RL, cellulose acetate phthalate, ethyl celluloses, hydroxypropyl methyl celluloses, hydroxypropyl celluloses, hydroxypropyl ethyl celluloses and the like.

In some embodiments, the formulations of the invention are in the form of pellets. Examples of such formulations include those containing pellets that contain a layer of active compound on top of an inert core, for example, a sugar sphere, and a surface coating containing one or more release rate controlling polymers. In other embodiments, the formulations are in the form of capsules, e.g., hard or soft gelatin capsules and/or powder.

In some embodiments, the formulations of the invention are in the form of tablets. The percentage by weight of active compound in the representative formulations of this type is from about 0.3% to about 25%, preferably from about 0.3% to about 15%. In some embodiments, the percentage by weight of active compound in the representative formulations of this type are about 1% to about 25%, preferably from about 2% to about 15%. Nonlimiting examples of such tablets are co-compressed tablets, e.g., “tablet-in-tablet” and matrix tablets.

The co-compressed tablet can include a core and-an-outer compressed coat. Either or both of the core and the outer compressed coat can contain active compound and/or one or more release rate controlling polymers. In some embodiments, the dosage form is a co-compressed tablet wherein both the core and the outer compressed coat contain active compound, and at least one release rate-controlling polymer, one of which is preferably a hydroxypropyl methyl cellulose. Preferred matrix forming polymers include a hydroxypropyl methylcellulose selected from Methocel™ K4M, Methocel™ K15M, Methocel™ K100M, Methocel™ E10M, Methocel™ E10M, Methocel™ E4M, Methocel™ K4M, Methocel™ K100LV, Methocel™ E50LV, Methocel™ E5, Methocel™ E6, Methocel™ E15LV or a combination of two or more thereof.

In some embodiments, the tablet is a matrix tablet. The matrix forming composition can contain waxes, gums, polyethylene oxides, carbapols, hydroxypropyl methylcelluloses, hydroxypropyl celluloses, hydroxyethyl celluloses, polymethacrylates or other release rate controlling polymers as described herein. In some embodiments, such matrix tablets are prepared by blending the active compound and the matrix-forming polymer together, and compressing the blend.

In some embodiments, the tablet is a matrix tablet that includes a wax matrix. Such tablets can be prepared, for example, by melting a wax such as carnauba wax, cetostearyl alcohol or fatty acids, or combinations thereof, and adding active compound along with a filler, such as microcrystalline cellulose as well as other excipients, fillers, lubricants and the like, and allowing the mixture to cool. The formulations prepared can be optionally coated with or contain one or more water-soluble or release rate controlling control release polymers. The wax can be present in the formulation in a total amount by weight of, for example, from about 10% to about 60%, preferably from about 20% to about 40%. A wide variety of suitable waxes is amenable to the present invention. Nonlimiting examples of such waxes include carnauba wax, cetostearyl alcohol, fatty acids, or a mixture or two or more thereof. The matrix tablet also can contain one or more release rate-controlling polymers as described herein.

In some embodiments, the matrix tablet is a tablet that includes a polyethylene oxide matrix, for example and not limitation, polyethylene oxide resins such as SENTRY POLYOX™ (Union Carbide Corporation) or equivalents. Suitable POLYOX's include POLYOX™ WSR N-10, N-60 K, WSR-1105N, or WSR 303. The POLYOX™ can have a molecular weight in the range of, for example, 100,000 to 7,000,000, or 900,000 to 5,000,000. The polyethylene oxide can be present in the formulation in a total amount by weight of, for example, from about 5% or about 10% to about 40%, or about 75% preferably from about 5% to about 40% or from about 10% to about 20% of the formulation. The matrix tablet also can contain one or more release rate-controlling polymers as described herein.

In some embodiments, the matrix tablet is a tablet that includes one or more release rate controlling polymers as described herein as the matrix forming polymer. In some embodiments, such tablets include one or more matrix forming hydroxypropyl methyl celluloses as described herein as the matrix forming polymer. In some preferred embodiments, it is advantageous to use a high viscosity hydroxypropyl methylcellulose such as Methocel™ K4M at an amount by weight of, for example, from about 15% to about 70%, preferably from about 18% to about 50%. Other high viscosity polymers can also be used such as, for example, Methocel™ K15M, Methocel™ K100M, or Methocel™ E4M and the like. In some embodiments, a low viscosity hydroxypropyl methylcellulose can be used, such as Methocel™ E5OLV, Methocel™ E5, Methocel™ E6, or Methocel™ E15LV or combinations thereof and the like. In certain embodiments, both a high viscosity and a low viscosity hydroxypropyl methylcellulose can be used in the matrix. In some embodiments, when the low density hydroxypropyl methylcelluloses is present in a range of from about 15% to about 70%, preferably from about 25% to about 50%, the high density hydroxypropyl methylcellulose is present in an amount by weight of from about 20% to about 50%.

In general, the active compound or ingredient can be contained within any layer of a dosage form of the invention, and sustained release of the active compound can be achieved by the use of a release rate controlling polymer either contained within the layer containing the active compound, or in any layer encompassing the layer containing the active compound, for example an enteric coating. Such an enteric coating can also be applied to pellets, beads or spheroids containing active compound, or the active compound can be contained within the enteric coating itself.

In some embodiments of the matrix tablet formulations of the invention, the active compound is present in an amount by weight of from about 0.02% to about 16%, preferably from about 0.02% to about 4%.

Tablets of the invention can be coated with water-soluble film coat(s), coloring agents, or coated with pH dependent or pH independent polymers to further control the rate of release of active compound. In some embodiments, the tablets are coated with a subcoat, an enteric coating or an overcoating, or any combination thereof. In some preferred embodiments, the tablets of the formulations of the invention are coated with film.

The present inventions provides methods and/or processes for preparing sustained-release formulations comprising 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salts thereof, structurally related compounds, and/or metabolites. In one embodiment, a composition comprising the active ingredient with at least one rate controlling polymer and at least one organic acid is compressed for a time and under conditions effective for forming a tablet thereof. In some embodiments, the tablet is further coated, e.g., with film.

In another embodiment, the active ingredient is mixed with at least one release rate controlling polymer and a least one organic acid thereby forming a blend. The blend can be further compressed for a time and under conditions to form a tablet. In some embodiments, the tablet is further coated, e.g., with film. In a preferred embodiment, the blend is a dry blend.

In some embodiments, the formulations are prepared by roller compaction. For example, tablets can be prepared by granulation followed by milling. In some embodiments, the active ingredient, filler (e.g., microcrystalline cellulose) and polymer (e.g., hydroxypropylmethylcellulose) are granulated and then milled. The milled granules are then mixed with additional excipients, such as, for example, citric acid and magnesium stearate.

Also included in accordance with the present invention are any of the numerous technologies that exist for attaining sustained release oral formulations including those described above, as well as micro and macroencapsulation, fibers, matrices both polymeric (high density and low density) and non-polymeric, foams, liposomes, micelles, gels, physically dispersed drug in polymeric, porous, slightly porous or non-porous matrices, adsorption onto ion exchange resins, mixing with or adsorption onto chemically or biologically degradable matrices and the like. The active compound can be formulated in such a way that the drug achieves a single maximal concentration or can be formulated so that the drug is pulsed in two or more peaks. Oral delivery can be via way of liquid or solid dosage form. Liquid dosage forms include syrups, suspensions, emulsions, elixirs, and the like. The liquid carrier can include an organic or aqueous base and can be further modified with suitable pharmaceutical additives such as solubilizers, emulsifiers, buffers, preservatives, sweeteners, flavoring agents, suspending agents, thickening agents, colorants, viscosity regulators, stabilizers or osmoregulators, or combinations thereof. The aqueous carrier can also contain, for example, polymeric substances or oils.

The present invention also provides immediate release dosage forms. Immediate release dosage forms of the present invention can comprise the active ingredient, for example, 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salts thereof, structurally related compounds, or metabolites. As in the sustained release formulations, in some embodiments, the active ingredient is micronized. Preferably, the immediate release formulations are substantially free of base.

In a preferred embodiment, the immediate release formulation comprises the active ingredient, at least one filler and at least one lubricant. The formulations of the invention additionally can include any of a variety of materials that confer beneficial properties to the formulation. Such materials include, for example, solubility modifiers such as surfactants such as, for example, sodium lauryl sulfate, acidic compounds, fillers, lubricants, antioxidants, pH modifiers, chelating agents, disintegrants, binders, stabilizers, excipients including water soluble excipients such as sugars, and water dispersing excipients such as microcrystalline cellulose, colloidal silicone dioxide, silicified microcrystalline cellulose and starch. The range of lubricant is typically from about, for example, 0.2% to about 5% by weight. In one embodiment of the present invention, the amount of lubricant in a 150 mg dosage form is from about 0.5 to about 1 mg. The range of filler can be, for example, from about 70% to about 99%, by weight. In one embodiment of the present invention, the amount of filler in a 150 mg dosage form is from about 80 to about 149 mg.

The immediate release dosage forms of the present invention can contain the active compound in any convenient percentage and part in relation to the other ingredients. Typically, the formulation comprises active ingredient in percentage of from about 0.05% to about 10%.

For example, in one embodiment, immediate release formulations comprise about 297 parts of filler, and about 1.5 parts of lubricant per part of active ingredient, e.g., 4-cyano-N-{(2R)-2-[4-(2,3-dihydro-benzo[1,4]dioxin-5-yl)-piperazin-1-yl]-propyl}-N-pyridin-2-yl-benzamide or pharmaceutically acceptable salt thereof.

In another embodiment, immediate release formulations comprise about 29 parts of filler, and about 0.15 parts of lubricant per part of active ingredient, e.g., 4-cyano-N-{(2R)-2-[4-(2,3-dihydro-benzo[1,4]dioxin-5-yl)-piperazin-1-yl]-propyl}-N-pyridin-2-yl-benzamide or pharmaceutically acceptable salt thereof.

In another embodiment, immediate release formulations comprise about 148 parts of filler, and about 0.75 parts of lubricant per part of active ingredient, e.g., 4-cyano-N-{(2R)-2-[4-(2,3-dihydro-benzo[1,4]dioxin-5-yl)-piperazin-1-yl]-propyl}-N-pyridin-2-yl-benzamide or pharmaceutically acceptable salt thereof.

In another embodiment, immediate release formulations comprise about 58 parts of filler, and about 0.3 parts of lubricant per part of active ingredient, e.g., 4-cyano-N-{(2R)-2-[4-(2,3-dihydro-benzo[1,4]dioxin-5-yl)-piperazin-1-yl]-propyl}-N-pyridin-2-yl-benzamide or pharmaceutically acceptable salt thereof.

The immediate release formulations contemplated by the present invention can be in any form suitable for administration to a mammal and are not limited to the examples presented herein.

The present invention provides methods and/or processes for preparing immediate release formulations comprising 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salts thereof, and/or metabolites thereof. In one embodiment, a composition comprising the active ingredient with at least one filler and at least one lubricant is compressed for a time and under conditions effective to form a tablet thereof. In some embodiments, the tablet is further coated, e.g., with film.

In some embodiments, the active ingredient is mixed with at least filler and a least one lubricant thereby forming a blend. The blend can be further compressed for a time and under conditions to form a tablet. In some embodiments, the tablet is further coated, e.g., with film.

In some embodiments, the formulations are prepared by roller compaction.

The immediate release dosage forms like the sustained release dosage forms can be, for example, in the form of coated pellets, spheres, capsules, powder, or tablets.

Thus, in accordance with the present invention there are provided sustained release and immediate release dosage forms, including oral and non-oral sustained release dosage formulations. Accordingly, the present invention includes each of the numerous technologies that exist for immediate release non-oral dosage formulations. Delivery of active compound in accordance with the present invention can be via mucosal, vaginal, rectal, ocular, transdermal, intrauterine, routes and the like.

The present invention therefore provides, inter alia, dosage forms for 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salts thereof, structurally related compounds, and/or metabolites, methods for immediate delivery of 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salts thereof, structurally related compounds, and/or metabolites, and methods for sustained delivery of 4-cyano-N-{(2R)-2-[4-(2,3-dihydro-benzo[1,4]dioxin-5-yl)-piperazin-1-yl]-propyl}-N-pyridin-2-yl-benzamide, pharmaceutically acceptable salts thereof, structurally related compounds, and/or metabolites over an extended period of time. In some embodiments, administration of the dosage form is once every 24 hours, once every 12 hours, or once every 6 hours.

The dosage forms described herein facilitate the immediate or sustained release of active compounds in a mammal through many routes, including oral administration. In some preferred embodiments, the dosage forms include the compound 4-cyano-N-{(2R)-2-[4-(2,3-dihydro-benzo[1,4]dioxin-5-yl)-piperazin-1-yl]-propyl}-N-pyridin-2-yl-benzamide, preferably the hydrochloride salt thereof, commonly referred to as lecozotan.

In preferred embodiments of the present invention, oral sustained release dosage forms comprising the drug lecozotan and a pharmaceutically acceptable carrier are provided. In one aspect, sustained release dosage forms comprising lecozotan, such as those described herein, possess improved pharmacokinetic profiles as compared to immediate release dosage forms. When administered to a patient population, oral dosage forms comprising lecozotan achieve a mean maximum (e.g., peak) plasma concentration (Cₘₐₓ) of lecozotan in the patient population and a mean minimum plasma concentration (Cₘᵢₙ) of lecozotan in the patient population. Preferred sustained release dosage forms of the present invention will minimize the variance between the Cₘₐₓ and Cₘᵢₙ plasma levels in the patient population. In particular, preferred sustained release dosage forms of the present invention will minimize the variance between the maximum and trough plasma levels in the patient population.

As used herein peak or maximum concentration (Cₘₐₓ) of an active ingredient, such as lecozotan, in blood plasma, area under concentration vs. time curve (AUC) of an active ingredient, such as lecozotan, in blood plasma, and time to maximal plasma concentration (tₘₐₓ) of an active ingredient, such as lecozotan, in blood plasma are pharmacokinetic parameters known to one skilled in the art. (Applied Biopharmaceutics and Pharmacokinetics, Chapter 7; Scargle and Yu, 4th edition, 1999). Unless otherwise indicated, the pharmacokinetic parameters are measured at steady state in a fasting population. The concentration vs. time curve measures the concentration of active ingredient in blood serum of a subject verses time after oral administration of a dosage form. “Cₘₐₓ” is the maximum concentration of active ingredient in the blood serum of a subject after administration of the dosage form to the subject. “tₘₐₓ” is the time to reach maximum concentration of active ingredient in the blood serum of a subject following administration of the dosage form to a subject. The plasma drug concentration at any time following drug administration is referenced as Cₜᵢₘₑ, as in C₉ₕ or C₂₄ₕ. “C_{trough}” refers to the plasma drug concentration at the end of the dosing interval. The “plasma drug concentration” or “plasma concentration” is, generally expressed as mass per unit volume, typically nanograms per milliliter.

Persons of skill in the art appreciate that plasma drug concentrations obtained in individual subjects will vary due to interpatient variability in the many parameters affecting drug absorption, distribution, metabolism and excretion. For this reason, unless otherwise indicated, mean values obtained from groups of patients (“patient populations”) are used herein.

As used herein, the area under concentration vs. time curve (AUC) of active ingredient, e.g., lecozotan, in blood plasma is calculated according to the linear up/log down trapezoidal rule. The “delivery rate” or “rate of absorption” is estimated by comparison of the time (tₘₐₓ) to reach the maximum concentration (Cₘₐₓ). Both Cₘₐₓ and tₘₐₓ are analyzed using non-parametric methods. Comparisons of the pharmacokinetics of immediate and sustained release formulations are performed by analysis of variance (ANOVA). P<0.05 is considered significant. Results are given as mean values +/−SEM. Equivalence of a pharmacokinetic parameter generally refers to the 90% confidence interval of the ratio of the central values of the pharmacokinetic parameter of the test formulation to the reference formulation being contained within about 0.8 to about 1.25.

In one aspect of the present invention, the sustained release formulations will achieve a Cₘₐₓ in a patient and a C₂₄ in a patient wherein the mean ratio of Cₘₐₓ/C₂₄ in a patient population is from about 5:1 to about 1.1:1. In some preferred embodiments, the mean ratio of Cₘₐₓ/C₂₄ in a patient population is from about 3:1 to about 1.1:1, from about 2.8:1 to about 1.1:1, from about 2.5 to about 1.1:1, from about 2.3:1 to about 1.1:1 or even from about 2:1 to about 1.1:1 or from about 1.5:1 to about 1.1:1. Accordingly, in certain embodiments, the mean ratio of Cₘₐₓ/C₂₄ in a patient population is about 5. In other embodiments, the mean ratio of Cₘₐₓ/C₂₄ in a patient population is about 3, about 2.8, about 2.5 or even about 2.3, about 2, or about 1.5. In certain embodiments, these Cₘₐₓ and C₂₄ values are measured after administration of a single dose of the drug to a patient. In some embodiments, a 5 mg single dose of the drug is administered. In other embodiments, these Cₘₐₓ and C₂₄ values are measured at steady state in a fasting population.

In one aspect of the present invention, the sustained release formulations will achieve a Cₘₐₓ in a patient and a C₂₄ in a patient wherein the mean ratio of Cₘₐₓ/C₂₄ in a patient population is from about 2.4:1 to about 1.1:1, from about 2.3:1 to about 1.1:1, from about 2:1 to about 1.1:1, from about 1.9 to about 1.1:1, from about 1.5:1 to about 1.1:1 or from about 1.25:1 to about 1.1:1. Accordingly, in certain embodiments, the mean ratio of Cₘₐₓ/C₂₄ in a patient population is about 2.3. In other embodiments, the mean ratio of Cₘₐₓ/C₂₄ in a patient population is about 2, about 1.9, about 1.5 or even about 1.25. In one aspect, these Cₘₐₓ and C₂₄ values are measured at steady state in a fasting population, e.g., after 28 daily doses of the drug to a patient. In some embodiments, the drug is administered at a daily dose of 10 mg. In other embodiments, these Cₘₐₓ and C₂₄ values are measured after administration of a single dose of the drug to a patient.

In some embodiments, the variation between Cₘₐₓ and C₁₂ or C₂₄ in a patient population can be characterized as a percent variation. For example, in certain aspects, the mean ratio of Cₘₐₓ/C₂₄ or Cₘₐₓ/C₁₂ in a patient population that has been administered lecozotan will vary no more than about 250%. In other embodiments, the mean ratio of Cₘₐₓ/C₂₄ or Cₘₐₓ/C₁₂ in a patient population will vary no more than about 100%, about 50%, about 30%, or even about 25%. In some embodiments, the Cₘₐₓ and C₁₂ or C₂₄ are measured at steady state. In other embodiments, the Cₘₐₓ and Cₘᵢₙ are measured after administration of a single dose of the drug to a patient.

In certain aspects of the present invention, the sustained release formulations will achieve a mean Cₘₐₓ in a patient population and a mean C₂₄ in a patient population wherein the ratio of mean Cₘₐₓ/mean C₂₄ is from about 5:1 to about 1.1:1. In some preferred embodiments, the ratio of mean Cₘₐₓ/mean C₂₄ is from about 2.8:1 to about 1.1:1, from about 2:4 to about 1.1:1, from about 2.3 to about 1.1:1, from about 2:1 to about 1.1:1, from about 1.5:1 to about 1.1:1 or even from about 1.25:1 to about 1.1:1. Accordingly, in certain embodiments, the ratio of mean Cₘₐₓ/mean C₂₄ is about 5, about 2.8, about 2.4, about 2.3, about 2, about 1.5, or even about 1.25. In one aspect, these Cₘₐₓ and C₂₄ values are measured after administration of a single dose of the drug to a patient. In some embodiments, a 5 mg single dose of the drug is administered. In one aspect, these Cₘₐₓ and C₂₄ values are measured at steady state in a fasting population, e.g., after 28 daily doses of the drug to a patient.

In one aspect of the present invention, the sustained release formulations will achieve a mean Cₘₐₓ in a patient population and a mean C₁₂ in a patient population wherein the ratio of mean Cₘₐₓ/mean C₁₂ is from about 3:1 to about 1.1:1. In some preferred embodiments, the ratio of mean Cₘₐₓ/mean C₁₂ is from about 2.3:1 to about 1.1:1, from about 2:1 to about 1.1:1, from about 1.9 to about 1.1:1, from about 1.5:1 to about 1.1:1 or from about 1.25:1 to about 1.1:1. Accordingly, in certain embodiments, the ratio of mean Cₘₐₓ/mean C₁₂ is about 2.3. In other embodiments, the ratio of mean Cₘₐₓ/mean C₁₂ is about 2, about 1.9, about 1.75 or even about 1.5, about 1.4, about 1.3, about 1.25, about 1.2, or about 1.1. In some embodiments, the Cₘₐₓ and C₁₂ are measured at steady state. In other embodiments, the Cₘₐₓ and C₁₂ are measured after administration of a single dose of the drug to a patient. In some embodiments, the drug is administered at a dosage of 5 mg.

Certain preferred sustained release formulations maintain therapeutic levels of lecozotan over a 24 hour dosing period, thus providing for once daily dosing. Preferred sustained release formulations also reduce the incidence or severity of side effects associated with lecozotan therapy. The adverse side effects include headache, dizziness, paresthesia, abnormal vision, tinnitus, or a combination thereof.

A decreased incidence of side effects refers to a reduced incidence of side effects in a patient population, and not to a total absence of side effects, when measured in a comparable population consuming an immediate release formulation of lecozotan.

Preferably, the sustained release formulations that have an improved pharmacokinetic profile have a dissolution profile wherein no more than about 40% of lecozotan is released at about 2 hours of measurement, and from about 50 to 85% of lecozotan is released at about 12 hours of measurement.

The dissolution of the sustained release tablets or oral dosage forms is determined as directed in the USP, using Apparatus II (paddles), at 50 rpm, in 900 milliliters of USP pH 6.8 phosphate buffer at 37° C. A sinker is used for each tablet or dosage form. A filtered sample of the dissolution medium is taken at the time(s) specified. The amount of active ingredient dissolved is determined by chromatographing the sample, along with a standard of known concentration, on a reverse-phase high performance liquid chromatography column. The concentration of active ingredient in each sample is determined by comparing the peak responses of the sample chromatogram with the peak responses of the standard chromatograms obtained concomitantly.

Certain sustained release formulations preferably reduce peak plasma levels of lecozotan without have a substantial impact on either Cₘᵢₙ (e.g., trough) levels of lecozotan or the extent of lecozotan absorption in the patient. Preferably, sustained release formulations achieve certain pharmacokinetic parameters when compared to an immediate release dosage form. One example of such an immediate release formulation is lecozotan, blended in microcrystalline cellulose, such as Avicel® brand from FMC Corporation, lactose monohydrate and magnesium stearate that results in greater than 75% dissolution of the active ingredient in less than 0.25 hours in a 0.1 N HCl solution.

Certain sustained release formulations of the present invention preferably achieve a mean Cₘₐₓ in a patient population that is lower than the mean Cₘₐₓ achieved in a patient population administered an immediate release dosage form. Preferably, mean peak plasma levels are reduced by at least about 10%, more preferably at least about 20%. In some embodiments, mean peak plasma levels are reduced by at least about 30% or more. In some embodiments, mean peak plasma levels are reduced by greater than 50%. Preferably, a single dose of a 5 mg oral dosage formulation achieves a mean Cₘₐₓ in a patient population that is about 100 ng/ml or lower. In another aspect of the present invention, multiple daily doses of 10 mg of lecozotan achieve a mean Cₘₐₓ in a patient population that is about 350 ng/ml. In some embodiments, the Cₘₐₓ is measured at steady state. In other embodiments, the Cₘₐₓ is measured after administration of a single dose of the drug to a patient.

Certain sustained release formulations of the present invention preferably achieve a mean tₘₐₓ in a patient population that is greater than the mean tₘₐₓ achieved in a patient population administered an immediate release dosage form. Preferably, tₘₐₓ is doubled, tripled, or even quadrupled. In certain embodiments of the present invention, the mean tₘₐₓ achieved in a patient population administered a sustained release dosage form of lecozotan is from about 4 to about 8 hours.

Sustained release formulations of the present invention preferably achieve a mean C_{trough} in a patient population that is substantially equivalent to the C_{trough} achieved in a patient population administered an immediate release dosage form. Maintaining comparable trough levels to those obtained with immediate release dosage forms maintains the therapeutic efficacy of the active ingredient. A mean C_{trough} of at least 80% should be achieved with the preferred sustained release formulations of the present invention over a 24-hour interval when compared to immediate release dosage form over a 12-hour interval. In some embodiments, the mean C_{trough} is at least about 90% or at least about 95% when compared to immediate release dosage form.

In order to maintain the therapeutic efficacy, it is also preferred that the total amount of lecozotan absorbed from the sustained release dosage form (AUC) is substantially equivalent to the total amount of lecozotan absorbed from an immediate release formulation over a 24 hour dosing interval. Accordingly, in preferred embodiments, the sustained release dosage forms achieve a mean AUC in a patient population that is substantially equivalent to the mean AUC achieved in a patient population administered an immediate release dosage form. For example, a mean AUC of at least about 80% should be achieved with the preferred sustained release formulations of the present invention when compared to immediate release dosage form over a 24 hour interval. In some embodiments, the mean AUC is at least about 90% or at least 95% when compared to immediate release dosage form over a 24-hour interval. The AUC should be no more than about 125% of the AUC of an immediate release formulation.

The present invention provides methods comprising administering the sustained release dosage forms to a patient. In certain embodiments, the sustained release dosage forms are administered to treat Alzheimer's disease in a patient. In some embodiments, the methods of administering sustained release dosage forms also include a step of identifying a patient at risk at adverse side effects through administration of lecozotan. In an exemplary embodiment of the present invention, to identify subject patients for treatment according to the methods of the invention, accepted screening methods can be employed to determine the status of Alzheimer's disease in a subject. In certain aspects, a routine medical history can be taken to determine whether the patient already suffers from or is prone to suffer from one or more of the side effects associated with administration of immediate release dosage forms of lecozotan. A patient that already suffers from one or more of these side effects or is prone to suffer from one or more of these side effects can be classified as a patient at risk of adverse effects through administration of lecozotan. In other aspects, an electrocardiogram can be performed to determine if the patient has any cardiac abnormalities, e.g., slow heart rate, that may indicate that he or she will be better treated with a formulation that has a reduced Cₘₐₓ. These and other routine methods allow the clinician to identify patients in need of therapy using the methods and formulations of the invention.

Preferred sustained release formulations of the present invention achieve certain mean pharmacokinetic parameters in a patient population. The patient population is selected in accordance with standard procedures for performing randomized studies. The patient population comprises at least 12 people selected without prior knowledge of the manner in which drugs of this class would be metabolized in them.

### EXAMPLES

### Example 1

### Identification of Metabolites of 4-cyano-N-{(2R)-2-[4-(2,3-dihydro-benzo[1,4]dioxin-5-yl)-piperazin-1-yl]-propyl}-N-pyridin-2-yl-benzamide

Four metabolites referred to as M8, M11, M12, and M13 were isolated by solvent (ethyl acetate containing 10% methanol) extraction followed by semi-preparative HPLC. The semi-preparative HPLC separation was conducted on a xTerra C18 column (7.8×300 mm, 10 μm), and a gradient of acetonitrile/water containing 10 mM ammonium acetate (pH=4.5) was used as the mobile phase.

The structures of the metabolites were determined based on NMR and mass spectra data. For NMR, all samples were dissolved in CD₃CN. For sample M11, about 10% D₂O was added to increase solubility. Proton and COSY data were acquired on all samples. For the samples containing M11 and M12, HSQC and HMBC data were also acquired to determine the structures.

M11, M12 and M13 metabolites were formed through hydroxylation at the dihydrobenzo-[1,4]dioxin-piperazine moiety. The NMR studies were conducted to determine the locations of the hydroxylation in these metabolites and to confirm the structure of M8.

M11: 1D proton spectrum of M11 showed two aromatic protons of the 2,3-dihydro-benzo[1,4]dixon moiety instead of 3 as in the parent compound, indicating the hydroxylation occurred on the benzene ring. The two proton signals are doublets suggesting the hydroxylation occurred either on C₆ or C₈ positions. To distinguish the two region isomers, a 1D NOE experiment was carried out, since relatively strong NOE correlations between the benzene proton H₆ and the piperazine protons are expected for C₈ hydroxylation, but not for C₆ hydroxylation. Such NOE correlations were indeed observed in the 1D NOE experiment. Therefore the structure of M11 is as shown below where the hydroxyl group is on C₈ of the 2,3-dihydro-benzo[1,4]dixon moiety.

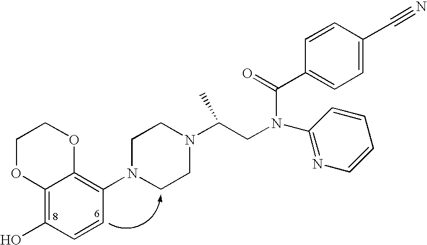

M12: 1D proton spectrum of M12 was more complicated than expected for the metabolite. A careful analysis of the spectrum however, suggested the sample contained isomers. Comparison of the proton spectrum of the M12 metabolite with that of the parent compound indicated that the aromatic moieties and the piperazine moiety are intact in M12. The protons of the 1,4-dioxin ring however, are quite different. Three methine signals are observed at 5.5, 5.15 sand 5.1 ppm. These methine protons integrated into one equivalent proton for the sample. The HSQC data showed that the carbon shifts of these methine groups are between 80 to 88 ppm, suggesting the hydroxylation on one of the dioxin methylenes. COSY spectrum showed that the down-filed methine proton correlates to the methylene protons of the dioxane ring, confirming the hydroxylation on the dioxane ring. The fact that more than two sets of signals were observed indicates chiral isomers existed in the sample. Whether the chiral isomers were generated by the enzymes or through racemization in the sample purification steps is not clear. Based on the NMR results the structure of M12 is as shown below:

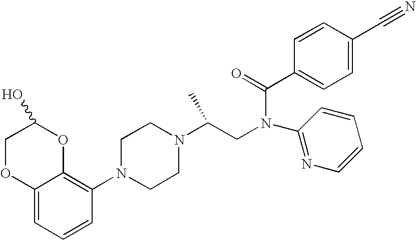

M13: Comparison of the proton spectrum of M13 with that of M12 suggests M12 and M13 are very similar. All aromatic protons observed in the parent compound were observed in M13 suggesting that the aromatic moieties are intact in the metabolite. It 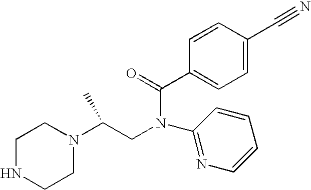 appeared that in M13, the hydroxylation also occurred on the dioxin ring. Similar to M12, M13 contained isomers as indicated by four methine protons observed at 5.5, 5.19, 5.10 and 4.86 ppm. It was noted that over time the intensity of these four methine signals changed, suggesting the ratio of the isomers have changed. Similar changes were observed in M12. Combined with the results from M12 analysis, it appeared that the observed NMR spectra of M12 and M13 might not represent the original components. The NMR analysis indicated that M12 and M13 were produced by hydroxylation on the dioxane ring, corresponding to 2 and 3 positions, respectively. M12 and M13 can rearrange, and both can be racemized.

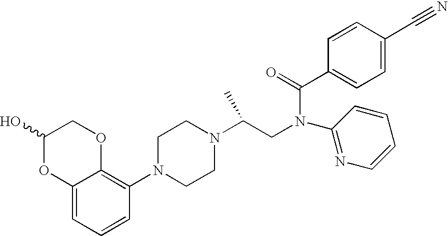

M8: Proton and COSY spectra of M8 were acquired for this sample. The data are consistent with the proposed structure for M8 based on MS/MS analysis performed by DSM. The pyridine moiety, the piperazine moiety and the cyano-propyl benzamide moieties are all intact. Compared with the parent compound, the only group missing is the 2,3-dihydro-benzo[1,4]dioxin moiety.

### Example 2

### Identification of Compounds Structurally Related to 4-cyano-N-{(2R)-2-[4-(2,3-dihydro-benzo[1,4]dioxin-5-yl)-piperazin-1-yl]-propyl}-N-pyridin-2-yl-benzamide

Structurally related compounds represented by Formulas 2-9 were identified. The structurally related compounds were isolated from a preparation comprising 4-cyano-N-{(2R)-2-[4-(2,3-dihydro-benzo[1,4]dioxin-5-yl)-piperazin-1-yl]-propyl}-N-pyridin-2-yl-benzamide by preparative chromatography in the amounts of about 1 mg with the purity of about 90%. The structures were established by nuclear magnetic resonance spectroscopy, electrospray ionization mass spectrometry and determination of the number of exchangeable protons.

A preparation comprising 4-cyano-N-{(2R)-2-[4-(2,3-dihydro-benzo[1,4]dioxin-5-yl)-piperazin-1-yl]-propyl}-N-pyridin-2-yl-benzamide hydrochloride salt was further processed as follows. The starting material was converted to base by treatment with aqueous sodium hydroxide and ethyl acetate. The resulting ethyl acetate solution was dried azeotropically, diluted with heptane to give a 3:1 ethyl acetate heptane mixture and treated with silica gel. The resulting mixture was filtered and concentrated repeatedly to remove heptane. The base was treated in ethyl acetate solution with 1.0 equivalent of hydrogen chloride in ethyl acetate. The product was dissolved in hot denatured ethanol. The mixture was filtered and concentrated. The product was crystallized by cooling and isolated by filtration. The final wet cake was dried. This process reduced the levels of dimeric impurities of 4-cyano-N-{(2R)-2-[4-(2,3-dihydro-benzo[1,4]dioxin-5-yl)-piperazin-1-yl]-propyl}-N-pyridin-2-yl-benzamide.

### Example 3

### Representative Sustained Release Formulations of the Present Invention

| | Function of | Amount per Tablet (mg) | | |
|---|---|---|---|---|
| Ingredient | Ingredient | Fast | Medium | Slow |

Active Core:

| | | | | |
|---|---|---|---|---|
| 4-cyano-N-{(2R)- | Active | 5.0 | 5.0 | 5.0 |
| 2-[4-(2,3-dihydro- | | | | |
| benzo[1,4]dioxin- | | | | |
| 5-yl)-piperazin-1- | | | | |
| yl]-propyl}-N- | | | | |
| pyridin-2-yl- | | | | |
| benzamide | | | | |
| hydrochloride^{a} | | | | |
| Silicified | Filler | 169.0 | 94.0 | 88.75 |
| microcrystalline | | | | |
| cellulose | | | | |
| (ProSolv ® HD 90) | | | | |
| HPMC | Polymer | 50.0 | 125.0 | 37.5 |
| (Methocel ™ K4M | | | | |
| Premium CR) | | | | |
| HPMC | Polymer | — | — | 112.75 |
| (Methocel ™ K100M | | | | |
| Premium CR) | | | | |
| Mg Stearate NF | Lubricant | 1.0 | 1.0 | 1.0 |
| Citric Acid, | To improve | 25.0 | 25.0 | 5.0 |
| Anhydrous | release | | | |
| | rate in intestine | | | |
| Weight of Core | | 250 | 250 | 250 |
| (mg) | | | | |

Film Coating:

| | | | | |
|---|---|---|---|---|
| Opadry White | White Film | 7.5 | 7.5 | 7.5 |
| (YS-1- | | | | |
| 18202A) | | | | |
| Opadry Clear (YS- | Clear Film | 1.25 | 1.25 | 1.25 |
| 1- | | | | |
| 19025A) | | | | |
| Total Tablet | | 258.75 | 258.75 | 258.75 |
| Weight(mg) | | | | |

^{a}The amount of active ingredient may need to be adjusted according to its release potency.

### Example 4

### Representative Sustained Release Formulations of the Present Invention

| | | |
|---|---|---|
| | Function of | Amount per |
| Ingredient | Ingredient | Tablet (mg) |

| | | | |
|---|---|---|---|
| 4-cyano-N-{(2R)- | Active | 5.0 | 2.0 |
| 2-[4-(2,3-dihydro- | | | |
| benzo[1,4]dioxin- | | | |
| 5-yl)-piperazin-1- | | | |
| yl]-propyl}-N- | | | |
| pyridin-2-yl- | | | |
| benzamide | | | |
| hydrochloride^{a} | | | |
| Microcrystalline | Filler | 46.5 | 52.5 |
| cellulose | | | |
| (Avicel ® PH112) | | | |
| Fast-Flow Lactose | Filler | 100.00 | 100.00 |
| HPMC | Polymer | 55.00 | 55.00 |
| (Methocel ™ K4M | | | |
| Premium CR) | | | |
| HPMC | Polymer | 37.50 | 37.50 |
| (Methocel ™ | | | |
| K100LV Premium | | | |
| CR LH) | | | |
| Mg Stearate NF | Lubricant | 1.0 | 1.0 |
| Citric Acid, | To improve | 5.00 | 2.00 |
| Anhydrous | release | | |
| | Rate in intestine | | |
| Weight of Core | | 250 | 250 |
| (mg) | | | |
| Opadry White | White Film | 7.5 | 7.5 |
| (YS-1- | | | |
| 18202A) | | | |
| Opadry Clear | Clear Film | 1.25 | 1.25 |
| (YS-1- | | | |
| 19025A) | | | |
| Total Tablet Wt. | | 258.75 | 258.75 |
| (mg) | | | |

^{a}The amount of active ingredient may need to be adjusted according to its release potency.

### Example 5

### Representative Immediate Release Formulations of the Present Invention

0.5 mg Tablets

| | Claim | % | Input |
|---|---|---|---|
| Ingredient | (mg) | Wt/Wt | (mg/tablet) |
| Active ingredients micronized^{a,b} | 0.5 | 0.33 | 0.50 |
| Lactose Monohydrate, NF^{b} | | 79.17 | 118.75 |
| Microcrystalline cellulose, NF | | 20.00 | 30.00 |
| Magnesium stearate, NF | | .50 | 0.75 |
| Total | | 100.0 | 150.00 |

The active ingredient is 4-cyano-N-{(2R)-2-[4-(2,3-dihydro-benzo[1,4]dioxin-5-yl)-piperazin-1-yl]-propyl}-N-pyridin-2-yl-benzamide hydrochloride
^{a}The active moiety portion (free base) is theoretically 93% of 4-cyano-N-{(2R)-2-[4-(2,3-dihydro-benzo[1,4]dioxin-5-yl)-piperazin-1-yl]-propyl}-N-pyridin-2-yl-benzamide hydrochloride drug substance. Actual amounts added are based on the potency of the hydrochloride drug substance. Inputs listed in the table above are based on the weight of the active ingredient.
^{b}If the hydrochloride drug substance is not at 100% potency, adjustment to the drug substance input must be made with corresponding adjustment to the lactose monohydrate input.
^{c}Includes an excess quantity. Theoretical quantity is 0.075 Kg. 1.0 mg Tablets,

| | Claim | % | Input |
|---|---|---|---|
| Ingredient | (mg) | Wt/Wt | (mg/tablet) |
| Active ingredients micronized^{a,b} | 1.0 | 0.67 | 1.0 |
| Lactose Monohydrate, NF^{b} | | 78.83 | 118.25 |
| Microcrystalline cellulose, NF | | 20.00 | 30.00 |
| Magnesium stearate, NF | | 0.50 | 0.75 |
| Total | | 100.0 | 150.00 |

The active ingredient is 4-cyano-N-{(2R)-2-[4-(2,3-dihydro-benzo[1,4]dioxin-5-yl)-piperazin-1-yl]-propyl}-N-pyridin-2-yl-benzamide hydrochloride
^{a}The active moiety portion (free base) is theoretically 93% of 4-cyano-N-{(2R)-2-[4-(2,3-dihydro-benzo[1,4]dioxin-5-yl)-piperazin-1-yl]-propyl}-N-pyridin-2-yl-benzamide hydrochloride drug substance. Actual amounts added are based on the potency of the hydrochloride drug substance. Inputs listed in the table above are based on the weight of the active ingredient.
^{b}If the hydrochloride drug substance is not at 100% potency, adjustment to the drug substance input must be made with corresponding adjustment to the lactose monohydrate input.
^{c}Includes an excess quantity. Theoretical quantity is 0.075 kg

2.5 mg Tablets

| | Claim | % | Input |
|---|---|---|---|
| Ingredient | (mg) | Wt/Wt | (mg/tablet) |
| Active ingredients micronized^{a,b} | 2.5 | 1.67 | 2.50 |
| Lactose Monohydrate, NF^{b} | | 77.83 | 116.75 |
| Microcrystalline cellulose, NF | | 20.00 | 30.00 |
| Magnesium stearate, NF | | .50 | 0.75 |
| Total | | 100.0 | 150.00 |

The active ingredient is 4-cyano-N-{(2R)-2-[4-(2,3-dihydro-benzo[1,4]dioxin-5-yl)-piperazin-1-yl]-propyl}-N-pyridin-2-yl-benzamide hydrochloride
^{a}The active moiety portion (free base) is theoretically 93% of 4-cyano-N-{(2R)-2-[4-(2,3-dihydro-benzo[1,4]dioxin-5-yl)-piperazin-1-yl]-propyl}-N-pyridin-2-yl-benzamide hydrochloride drug substance. Actual amounts added are based on the potency of the hydrochloride drug substance. Inputs listed in the table above are based on the weight of the active ingredient.
^{b}If the hydrochloride drug substance is not at 100% potency, adjustment to the drug substance input must be made with corresponding adjustment to the lactose monohydrate input.
^{c}Includes an excess quantity. Theoretical quantity is 0.075 kg

5.0 mg Tablets

| | Claim | % | Input |
|---|---|---|---|
| Ingredient | (mg) | Wt/Wt | (mg/tablet) |
| Active ingredients micronized^{a,b} | 5.0 | 3.33 | 5.0 |
| Lactose Monohydrate, NF^{b} | | 76.17 | 114.25 |
| Microcrystalline cellulose, NF | | 20.00 | 30.00 |
| Magnesium stearate, NF | | .50 | 0.75 |
| Total | | 100.0 | 150.00 |

The active ingredient is 4-cyano-N-{(2R)-2-[4-(2,3-dihydro-benzo[1,4]dioxin-5-yl)-piperazin-1-yl]-propyl}-N-pyridin-2-yl-benzamide hydrochloride
^{a}The active moiety portion (free base) is theoretically 93% of 4-cyano-N-{(2R)-2-[4-(2,3-dihydro-benzo[1,4]dioxin-5-yl)-piperazin-1-yl]-propyl}-N-pyridin-2-yl-benzamide hydrochloride drug substance. Actual amounts added are based on the potency of the hydrochloride drug substance. Inputs listed in the table above are based on the weight of the active ingredient.
^{b}If the hydrochloride drug substance is not at 100% potency, adjustment to the drug substance input must be made with corresponding adjustment to the lactose monohydrate input.
^{c}Includes an excess quantity. Theoretical quantity is 0.075 kg

### Example 6

### Representative Manufacturing Directions for Representative Immediate Release Tablets

1. Dispense the lactose monohydrate and microcrystalline cellulose into suitable containers.
2. Dispense the 4-cyano-N-{(2R)-[4-(2,3-dihydro-benzo[1,4]dioxin-5-yl)-piperazin-1-yl]-propyl}-N-pyridin-2-yl-benzamide hydrochloride into a suitably sized tumbler mixing bowl. Add a small portion of the dispensed lactose monohydrate and mix into a tumbling mixer.
3. Pass the pre-blend from step 2, followed by the microcrystalline cellulose, through a 500 μm screen into a suitably sized tumbler mixer bowl. Mix.
4. Transfer the pre-blend from step 3 into a suitably sized tumbler mixer bowl. Pass the remaining lactose monohydrate through a 500 μm screen into the mixing bowl. Mix.
5. Weigh the blend and calculate the amount of magnesium stearate required for the batch. Dispense the magnesium stearate into a suitable container, and mix with a portion of the blend from step 4.
6. Pass this pre-mix through a 500 μm screen and into the remaining blend in the mixing bowl. Mix the final blend.
7. Compress the blend from step 6 using a suitable compression machine fitted with appropriate tooling, to produce tablets with the required weight and hardness.
8. De-dust, weight check, and visually inspect the finished tablets.

### Example 7

### Single Dose Study Between Three Sustained Release Formulations and an Immediate Release Formulation of Lecozotan

This study was a single-dose, randomized, 4-period, crossover, bioequivalence study between three modified release formulations and an immediate release formulation of lecozotan in healthy subjects. Doses were administered orally after an overnight fast of at least 10 hours. Formulations used in this study were 5 mg of an immediate release (IR), 5 mg of a modified fast-release formulation (MR (fast)), 5 mg of a modified medium-release formulation (MR (medium)), and 5 mg of a modified slow-release formulation (MR (slow)). These formulations are provided in Example 3. Blood samples for lecozotan analysis were obtained within 2 hours of test article administration and at 0.5, 1, 1.5, 2, 3, 4, 6, 8, 10, 12, 16, 24, 30, 36 and 48 hours after test article administration.

Plasma concentration data and PK parameters of lecozotan were compared between the four formulations using an analysis of variance for a four-period crossover study. Additionally, the geometric mean (log-transformed) relative bioavailability of the maximum observed concentration (Cₘₐₓ) of lecozotan, area under the concentration-time curve (AUC) and AUC up to the time of the last quantifiable concentration (AUC_{T}) and their 90% confidence limits were estimated to determine the magnitude of difference in these parameters between the 4 formulations. The lecozotan immediate release tablet was used as the reference treatment. The 90% confidence limits for parameter estimates were constructed on the log scale using the 2 1-sided tests procedure. The 4 treatments (formulations) were judged to be bioequivalent if the 90% confidence limits fell within the interval (0.80, 1.25).

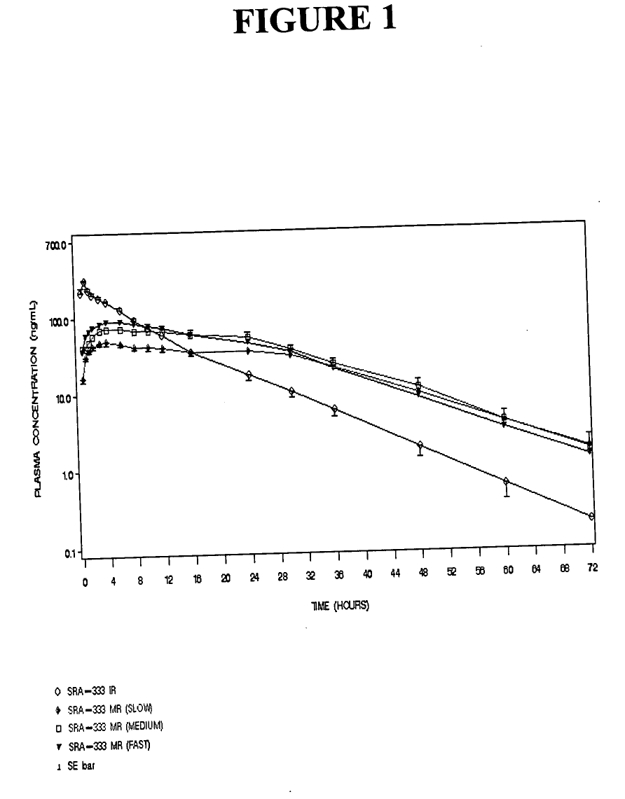

A total of 20 subjects contributed pharmacokinetic data for this analysis. FIG. 1 provides mean concentrations of lecozotan versus time for the four formulations. As shown by FIG. 1, the mean Cₘₐₓ for the IR formulation is 278.5 ng/mL. The mean values for the modified release formulations are below 100 ng/mL. The MR (fast) and MR (medium) formulations have Cₘₐₓ values of 99.7 and 95.1 ng/mL, respectively. The MR (slow) formulation has a Cₘₐₓ value of 58.4 ng/mL. The Tₘₐₓ for the IR formulation occurs at 0.88 hours. Tₘₐₓ values for the MR (fast), MR (medium) and MR (slow) occur at 5.63, 8.83 and 7.90 hours, respectively.

AUC values for the IR, MR (fast) and MR (medium) formulations range from 2058.8 to 2246.8 ng h/mL. The AUC value for the MR (slow) formulation is 1565.0 ng h/mL. The mean AUC ratio of MR to the IR formulations is above 100% for the MR (fast) and MR (medium). The mean ratio of AUC for MR (slow) to IR is 79.3%. The mean ratio of Cₘₐₓ lecozotan concentrations at 24 hours (Cₘₐₓ/C_{24 h} ratio) ranges from 23 for the IR formulation to values of 2.8, 2.3 and 3.5 for the MR (fast), MR (medium), MR (slow) formulations, respectively.

### Example 8

### Multiple Dose Study Between Three Sustained Release Formulations and an Immediate Release Formulation of Lecozotan

This study was a randomized, double-blind, placebo-controlled study to assess safety, tolerability, pharmacokinetic and pharmacodynamic study of 1) ascending multiple-doses of 0.5, 1, 2.5 and 5 mg lecozotan administered orally as a twice daily regimen (q12h) to patients with mild to moderate Alzheimer's disease (AD; age range=53 to 80 yrs) using the immediate-release (IR) formulation and 2) multiple doses of 10 mg administered orally as once-daily regimen (QD) to patients with mild to moderate Alzheimer's disease using the sustained-release (SR) formulation.

Part 1: IR Formulation

Six subjects received lecozotan and 2 received placebo in the 0.5, 1 and 2.5 mg dose groups (cohorts 1-3) whereas 12 subjects received lecozotan and 4 received placebo in the 5 mg dose group (cohort 4). Blood samples for lecozotan analysis were obtained on days 1 and 28 within 2 hours of test article administration and at 0.5, 1, 1.5, 2, 4, 8, 24 (day 1 only) and 28 (day 28 only) hours after test article administration.

Part 2: SR Formulation

In this part of the study, 12 subjects received lecozotan (10 mg QD of SR formulation) and 4 received placebo. Blood samples for lecozotan analysis were obtained at predose (within 2 hours of test article administration), 0.5, 1, 1.5, 2, 4, 8, 24 hours post dose on day 1 and at predose, 1, 2, 4, 8, 24, 48 and 72 hours post dose on day 28.

A total of 11 AD patients (age range=52 to 90 yrs) contributed pharmacokinetic data for this analysis. After both single and multiple dose administration of the SR formulation, a plateau-like concentration vs. time profile was seen for all subjects up to a period of 24 hours, with peak/trough fluctuation (i.e. Cₘₐₓ/Cₘᵢₙ)=1.9 at steady-state. Lecozotan concentrations were seen to start declining after 24 hours. In part 1, mean tₘₐₓ for lecozotan with the IR formulation was seen to range from 1-1.5 hours after both single and multiple dose administration. In comparison, lecozotan mean tₘₐₓ values with the SR formulation after single and multiple dosing were 8 and 4 hrs, respectively. The dose dependent pharmacokinetic parameters Cₘₐₓ and AUC₀₋₂₄ after single dose administration of the SR formulation at a dose of 5 mg and 10 mg is as follows: 5 mg SR single dose: Cₘₐₓ=99.7 ng/mL, AUC₀₋₂₄=1845 ng hr/mL; 10 mg SR single dose: Cₘₐₓ=210.4 ng/mL, AUC₀₋₂₄=3738.3 ng hr/mL).

Based on pharmacokinetic modeling of single dose plasma concentration data obtained with 5 mg SR formulation, it was predicted that at steady state, a QD regimen of the SR formulation is predicted to achieve similar exposure (AUC₀₋₂₄), a lower Cₘₐₓ and a similar Cₘᵢₙ in comparison to the q12h regimen with the IR formulation. In agreement with these predictions, the mean lecozotan steady-state total daily exposure (AUC₀₋₂₄) was 5788 ng*hr/mL after administration of 5 mg q12h IR formulation and 6111 ng*hr/mL after administration of 10 mg QD SR formulation in AD patients. Mean lecozotan steady-state Cₘₐₓwas 450 ng/mL (model predicted=425 ng/mL) with 5 mg q12h IR regimen in comparison to the mean steady-state Cₘₐₓ of 353 ng/mL (model-predicted=319 ng/mL) with 10 mg QD SR formulation. Trough concentrations at the end of the dosing interval are 180 ng/mL and 184 ng/mL for 5 mg q12h of IR and 10 mg QD of SR formulations, respectively. Mean Cₘₐₓ/Cₘᵢₙ ratio at steady-state for 10 mg QD SR formulation was 1.91. Mean Cₘₐₓ/Cₘᵢₙ ratio at steady-state for 5 mg q12h of IR was 2.4. Mean tₘₐₓ with the 5 mg q12h IR regimen was 0.8 while mean tₘₐₓ with 10 mg QD SR formulation was 4.2.

### Example 9

### Adverse Effects

The following table demonstrates that the incidence of side effects was reduced following treatment with sustained release formulations of lecozotan as compared to the immediate release formulation of lecozotan.

Treatment of Emergent Adverse Events (TEAEs)
Immediate Release (IR) vs. Sustained Release Formulation

| | Lecozotan | | |
|---|---|---|---|
| | 5 mg | Lecozotan 5 mg | Lecozotan 5 mg |
| TEAE | IR | SR - fast | SR - medium |
| Headache | 1 | 0 | 0 |
| Dizziness | 9 | 0 | 1 |
| Paresthesia | 1 | 0 | 0 |
| Abnormal Vision | 2 | 0 | 0 |
| Tinnitus | 1 | 0 | 0 |
| Total | 14 | 0 | 1 |

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

All publications and patent documents cited above are hereby incorporated by reference in their entirety for all purposes to the same extent as if each were so individually denoted.

What is claimed:

1. A method comprising administering to a patient an oral dosage form that comprises lecozotan and a pharmaceutically acceptable carrier and achieves a maximum plasma concentration (Cₘₐₓ) in a patient and a 24 hour plasma concentration (C₂₄) in a patient, wherein a mean ratio of Cₘₐₓ/C₂₄ in a patient population is from about 5:1 to about 1.1:1.

2. The method of claim 1, wherein the Cₘₐₓ and C₂₄ values are measured after administration of a single dose of the oral dosage form to a patient.

3. The method of claim 2, wherein the oral dosage form comprises about 5 mg of lecozotan.

4. The method of claim 2, wherein the mean Cₘₐₓ in the patient population is about 100 ng/ml or lower.

5. The method of claim 1, wherein the mean ratio of Cₘₐₓ/C₂₄ in the patient population is from about 3:1 to about 1.1:1.

6. The method of claim 5, wherein the Cₘₐₓ and C₂₄ values are measured after administration of a single dose of the oral dosage form to a patient.

7. The method of claim 1, wherein the mean ratio of Cₘₐₓ/C₂₄ in the patient population is from about 2.8:1 to about 1.1:1.

8. The method of claim 1, wherein the mean ratio of Cₘₐₓ/C₂₄ in the patient population is from about 2.3:1 to about 1.1:1.

9. The method of claim 1, wherein the mean tₘₐₓ in the patient population is about 3.5 hours or greater.

10. The method of claim 1, wherein the mean tₘₐₓ in the patient population is about 5 hours or greater.

11. A method comprising administering to a patient an oral dosage form that comprises lecozotan and a pharmaceutically acceptable carrier and achieves a maximum plasma concentration (Cₘₐₓ) in a patient and a 24 hour plasma concentration (C₂₄) in a patient, wherein a mean ratio of Cₘₐₓ/C₂₄ in a patient population is from about 2.4:1 to about 1.1:1.

12. The method of claim 11, wherein the Cₘₐₓ and C₂₄ values are measured at steady state in a fasting population.

13. The method of claim 12, wherein about 10 mg of lecozotan is provided daily to the patient.

14. The method of claim 12, wherein the mean Cₘₐₓ is about 350 ng/ml.

15. The method of claim 11, wherein the mean ratio of Cₘₐₓ/C₂₄ in the patient population is from about 2.3:1 to about 1.1:1.

16. The method of claim 11, wherein the mean ratio of Cₘₐₓ/C₂₄ in the patient population is from about 2:1 to about 1.1:1.

17. The method of claim 11, wherein the mean ratio of Cₘₐₓ/C₂₄ in the patient population is from about 1.9:1 to about 1.1:1.

18. The method of claim 11, wherein the mean ratio of Cₘₐₓ/C₂₄ in the patient population is from about 1.5:1 to about 1.1:1.

19. A method comprising administering to a patient an oral dosage form that comprises lecozotan and a pharmaceutically acceptable carrier and achieves a mean maximum plasma concentration (Cₘₐₓ) in a patient population and a mean 24 hour plasma concentration (C₂₄) in a patient population, wherein a ratio of mean Cₘₐₓ/mean C₂₄ is from about 5:1 to about 0.5:1.

20. The method of claim 19, wherein the Cₘₐₓ and C₂₄ values are measured after administration of a single dose of the oral dosage form to a patient.

21. The method of claim 20, wherein the oral dosage form comprises about 5 mg of lecozotan.

22. The method of claim 19, wherein the ratio of mean Cₘₐₓ/mean C₂₄ is from about 2.8:1 to about 1.1:1.

23. The method of claim 19, wherein the ratio of mean Cₘₐₓ/mean C₂₄ is from about 2:1 to about 1.1:1.

24. The method of claim 19, wherein the ratio of Cₘₐₓ/mean C₂₄ is from about 1.5:1 to about 1.1:1.

25. A method comprising administering to a patient an oral dosage form that comprises lecozotan and a pharmaceutically acceptable carrier and achieves a mean maximum plasma concentration (Cₘₐₓ) in a patient population and a mean 12 hour plasma concentration (C₁₂) in a patient population, wherein a ratio of mean Cₘₐₓ/mean C₁₂ is from about 3:1 to about 0.5:1.

26. The method of claim 18, wherein the ratio of mean Cₘₐₓ/mean C₁₂ is from about 2:1 to about 1.1:1.

27. The method of claim 18, wherein the ratio of mean Cₘₐₓ/mean C₁₂ is from about 1.5:1 to about 1.1:1.

28. A method for minimizing an adverse side effect associated with the administration of lecozotan to a patient, comprising administering to a patient an oral dosage form that comprises lecozotan and a pharmaceutically acceptable carrier and achieves a maximum plasma concentration (Cₘₐₓ) in a patient and a 24 hour plasma concentration (C₂₄) in a patient, wherein a mean ratio of Cₘₐₓ/C₂₄ in a patient population is from about 5:1 to about 1.1:1.

29. The method of claim 28, wherein the Cₘₐₓ and C₂₄ values are measured after administration of a single dose of the oral dosage form to a patient.

30. A method for minimizing an adverse side effect associated with the administration of lecozotan to a patient, comprising administering to a patient an oral dosage form that comprises lecozotan and a pharmaceutically acceptable carrier and achieves a maximum plasma concentration (Cₘₐₓ) in a patient and a 24 hour plasma concentration (C₂₄) in a patient, wherein a mean ratio of Cₘₐₓ/C₂₄ in a patient population is from about 2.4:1 to about 1.1:1.

31. The method of claim 30, wherein the Cₘₐₓ and C₂₄ values are measured at steady state in a fasting population.

32. A method for minimizing an adverse side effect associated with the administration of lecozotan to a patient, comprising
identifying a patient at risk of an adverse side effect through administration of lecozotan; and
administering to a patient an oral dosage form that comprises lecozotan and a pharmaceutically acceptable carrier and achieves a maximum plasma concentration (Cₘₐₓ) in a patient and a 24 hour plasma concentration (C₂₄) in a patient, wherein a mean ratio of Cₘₐₓ/C₂₄ in a patient population is from about 5:1 to about 1.1:1.

33. The method of claim 32, wherein the Cₘₐₓ and C₂₄ values are measured after administration of a single dose of the oral dosage form to a patient.

34. The method of claim 32, wherein the adverse side effect is headache, dizziness, paresthesia, abnormal vision, tinnitus, or a combination thereof.

35. The method of claim 32, wherein the incidences of the adverse side effects are reduced.

36. A method for minimizing an adverse side effect associated with the administration of lecozotan to a patient, comprising
identifying a patient at risk of an adverse side effect through administration of lecozotan; and
administering to a patient an oral dosage form that comprises lecozotan and a pharmaceutically acceptable carrier and achieves a maximum plasma concentration (Cₘₐₓ) in a patient and a 24 hour plasma concentration (C₂₄) in a patient, wherein a mean ratio of Cₘₐₓ/C₂₄ in a patient population is from about 2.4:1 to about 1.1:1.

37. The method of claim 36, wherein the Cₘₐₓ and C₂₄ values are measured at steady state in a fasting population.

38. The method of claim 36, wherein the adverse side effect is headache, dizziness, paresthesia, abnormal vision, tinnitus, or a combination thereof.

39. The method of claim 36, wherein the incidences of the adverse side effects are reduced.

40. A method for administering lecozotan to a patient, the method comprising administering to the patient an oral dosage form that comprises lecozotan and a pharmaceutically acceptable carrier, wherein the dosage form exhibits an in vitro dissolution profile when measured in a USP type II dissolution apparatus at 50 rpm, in 900 ml of USP pH 6.8 phosphate buffer at 37° C. and a sinker is used for each dosage form, wherein:
no more than 40% of lecozotan is released at about 2 hours of measurement in said apparatus; and
from about 50% to about 85% of lecozotan is released at about twelve hours of measurement in said apparatus.

41. A method for administering lecozotan to a patient, the method comprises administering an oral dosage form that comprises lecozotan and a pharmaceutically acceptable carrier to a patient and
(i) achieves a mean Cₘₐₓ in a patient population that is less than the mean Cₘₐₓ obtained from administering an equivalent dose of lecozotan from an immediate release formulation to the patient population;
(ii) achieves a mean tₘₐₓ in a patient population that is greater than the tₘₐₓ obtained from administering an equivalent dose of lecozotan from an immediate release formulation to the patient population; and
(iii) achieves a curve of concentration of lecozotan over time, the curve having an area under the curve (AUC) in a patient population that is substantially the same as the AUC obtained from administering an equivalent dose of lecozotan from an immediate release dosage form to the patient population.

42. The method of claim 41, wherein the oral dosage form achieves a mean Cₘₐₓ of about 100 ng/ml or less.

43. The method of claim 42, wherein when the Cₘₐₓ is measured after administration of a single dose of the oral dosage form.

44. The method of claim 43, wherein the oral dosage form comprises about 5 mg of lecozotan.

45. The method of claim 41, wherein the oral dosage form achieves a mean tₘₐₓ of from about 4 hours to about 8 hours.

46. The method of claim 41, wherein the oral dosage form achieves a mean AUC that is from about 2000 to about 2500 ng h/mL.

47. The method of claim 46, wherein when the mean AUC is measured after administration of a single dose of the oral dosage form.

48. The method of claim 47, wherein the oral dosage form comprises about 5 mg of lecozotan.

49. The method of claim 41, wherein the oral dosage form achieves a mean Cₘₐₓ of less than 400 ng/mL.

50. The method of claim 49, wherein the oral dosage form achieves a mean Cₘₐₓ of about 350 ng/ml.

51. The method of claim 49, wherein when the Cₘₐₓ is measured at steady state in a fasting population.

52. The method of claim 51, wherein 10 mg of lecozotan is administered daily to the patient.

53. The method of claim 41, wherein the oral dosage form achieves a mean tₘₐₓ of from about 4 hours to about 8 hours.

54. The method of claim 41, wherein the oral dosage form achieves a mean AUC that is from about 5500 to about 6300 ng h/mL.

55. A method for minimizing an adverse side effect associated with the administration of lecozotan to a patient, the method comprises administering an oral dosage form that comprises lecozotan and a pharmaceutically acceptable carrier to a patient and
(i) achieves a mean Cₘₐₓ in a patient population that is less than the mean Cₘₐₓ obtained from administering an equivalent dose of lecozotan from an immediate release formulation to the patient population;
(ii) achieves a mean tₘₐₓ in a patient population that is greater than the tₘₐₓ obtained from administering an equivalent dose of lecozotan from an immediate release formulation to the patient population; and
(iii) achieves a curve of concentration of lecozotan over time, the curve having an area under the curve (AUC) in a patient population that is substantially the same as the AUC obtained from administering an equivalent dose of lecozotan from an immediate release dosage form to the patient population.

56. A method for minimizing an adverse side effect associated with the administration of lecozotan to a patient, comprising
identifying a patient at risk of an adverse side effect through administration of lecozotan; and
administering to the patient an oral dosage form that comprises lecozotan and a pharmaceutically acceptable carrier to the patient and
(i) achieves a mean Cₘₐₓ in a patient population that is less than the mean Cₘₐₓ obtained from administering an equivalent dose of lecozotan from an immediate release formulation to the patient population;
(ii) achieves a mean tₘₐₓ in a patient population that is greater than the mean tₘₐₓ obtained from administering an equivalent dose of lecozotan from an immediate release formulation to the patient population; and
(iii) achieves a curve of concentration of lecozotan over time, the curve having an area under the curve (AUC) in a patient population that is substantially the same as the AUC obtained from administering an equivalent dose of lecozotan from an immediate release dosage form to the patient population.

57. A method of treating Alzheimer's disease in a patient comprising administering a sustained release formulation of lecozotan to the patient.
